(19) United States
(12) Patent Application Publication
(10) Pub. No.: US 2004/0229219 A1
(43) Pub. Date: Nov. 18, 2004

(54) Method of inhibiting human metapneumovirus and human coronavirus in the prevention and treatment of severe acute respiratory syndrome (SARS)

(76) Inventors: William R. Gallaher, Pearl River, LA (US); Robert F. Garry, New Orleans, LA (US)

Correspondence Address: Mark E. Mahaffey, 8555 United Plaza Blvd., 5th Floor, Baton Rouge, LA, 70809, US

(21) Appl. No.: 10/834,666

(22) Filed: Apr. 29, 2004

Related U.S. Application Data
(60) Provisional application No. 60/466,978, filed on Apr. 30, 2003.

(51) Int. Cl. C07K 14/165; C12Q 1/70

(52) U.S. Cl. 435/5; 530/395

(57) ABSTRACT

The present invention relates to peptides that show significant antiviral activity against viral respiratory disease. More particularly, the invention relates to the use of peptides to inhibit membrane fusion and infection by human metapneumovirus and/or human coronavirus in the prevention and treatment of Severe Acute Respiratory Syndrome (SARS) or other severe respiratory diseases caused by theses agents. The peptides are derived from the known amino acid sequence of the fusion glycoproteins of each virus.

### CROSS-REFERENCE TO RELATED APPLICATION

This application claims the benefit of U.S. Provisional Application No. 60/466,978, filed Apr. 30, 2003, which is hereby incorporated by reference herein in its entirety.

### STATEMENT REGARDING FEDERALLY SPONSORED RESEARCH AND DEVELOPMENT

This invention was made with Government support under Grants No. AI-54238, No. AI-34764 and No. CA-08921 awarded by the National Institutes of Health, and Grant No. BC990847 awarded by the Department of Defense. The Government has certain rights in the invention.

### BACKGROUND OF THE INVENTION

A. Field of the Invention

The present invention relates to peptides that show significant antiviral activity. In certain embodiments, the invention relates to the design and use of peptides to inhibit membrane fusion and infection by human metapneumovirus and human coronavirus in the prevention and treatment of Severe Acute Respiratory Syndrome (SARS) or other severe respiratory diseases caused by theses agents.

B. Description of Related Art

SARS is a newly emerging infection which was first identified during an outbreak in southern China in March of 2003 (Drosten et al. “Identification of a Novel Coronavirus in Patients with Severe Acute Respiratory Syndrome,” N Engl J Med 2003 Apr. 10; Ksiazek et al. “A Novel Coronavirus Associated with Severe Acute Respiratory Syndrome,” N Engl J Med 2003 Apr. 10; Poutanen S M, et al. “Identification of Severe Acute Respiratory Syndrome in Canada.” N Engl J Med 2003 Mar. 31). By Apr. 16, 2003, over 3,235 individuals had been diagnosed with SARS, and over 161 SARS-related deaths had been recorded in 22 countries, on every continent except Antarctica. Air travel by infected individuals during the incubation period prior to the onset of symptoms greatly facilitated the spread of the infection to many countries, including the United States. The nature of the epidemic and the exact etiologic agent(s) of SARS are still under investigation. However, the illness does not appear to involve bacterial, fungal, or previously identified viral agents of human disease. Molecular amplification of nucleic acid sequences from patient samples revealed that a number of patients were infected contemporaneously by human metapneumovirus (MPV) and a new human coronavirus (CoV).

Through the end of 2003, the initial overall outbreak totaled approximately 8,098 cases of SARS worldwide, with an overall mortality of 9.6% (http://www.who.int/csr/sars/en/). The previously unrecognized SARS CoV has been demonstrated to have been the principal cause of the new disease (Drosten et al., 2003; Poutanen et al., 2003; Peiris et al. (2003). Coronavirus as a possible cause of severe acute respiratory syndrome. Lancet 361, 1319-25). (It is important to note that metapneumovirus was found in a substantial percentage of cases in China, to an extent greater than pure coincidence would indicate; thus, its role in increasing the severity of SARS cannot be ruled out.) In a remarkably short period of time, the entire genetic sequences of several strains of the novel SARS CoV were determined (Ksiazek et al., 2003; Marra et al. (2003). The genome sequence of the SARS associated coronavirus. Science 300, 1399-1404; Rota et al. Characterization of a novel coronavirus associated with severe acute respiratory syndrome. Science. 2003 May 30; Wang et al. (2003). Gene sequence analysis of SARS-associated coronavirus by nested RT-PCR. Di Yi Jun Yi Da Xue Xue Bao 23, 421-3, each of which is hereby incorporated by reference herein in its entirety) and the cellular receptor, ACE-2, for the virion identified (Li et al. (2003). Angiotensin-converting enzyme 2 is a functional receptor for the SARS coronavirus. Nature 426, 450-454). Isolates of a similar CoV were obtained from civets and other animals that are trapped for food or medicine at live animal markets in Guangdong mainland China, the presumed epicenter of the SARS outbreak (Guan et al. (2004). Molecular epidemiology of the novel coronavirus that causes severe acute respiratory syndrome. Lancet 363, 99-104). SARS CoV or a closely related CoV also infects animals in the wild (Guan, Y., Zheng, B. J., He, Y. Q., Liu, X. L., Zhuang, Z. X., Cheung, C. L., Luo, S. W., Li, P. H., Zhang, L. J., Guan, Y. J., Butt, K. M., Wong, K. L., Chan, K. W., Lim, W., Shortridge, K. F., Yuen, K. Y., Peiris, J. S., and Poon, L. L. (2003). Isolation and characterization of viruses related to the SARS coronavirus from animals in southern China. Science 302, 276-278.) and appears to have entered the human population in the past (Zheng et al. (2004). SARS-related virus predating SARS outbreak, Hong Kong. Emerging Infectious Diseases. e-pub Jan. 16, 2004). Public health interventions such as surveillance, travel restrictions, and quarantines contained the spread of SARS in 2003 and appear to have stopped the spread of SARS after the appearance of a few new cases in 2004. It is unknown, however, whether these draconian containment measures can be sustained with each appearance of SARS in humans. Furthermore, this new and sometimes lethal CoV has potential as a bioterrorism threat. There are no antiviral agents which are known to be effective in the treatment of SARS, and no antiviral agents are known to be effective against either metapneumovirus or coronavirus in humans or animals.

Human MPV is a recently characterized agent of human respiratory infection that appears to be a member of the Paramyxoviridae family of viruses (van den Hoogen, B. G. et al. “Analysis of the genomic sequence of a human metapneumovirus,” Virology 2002 Mar. 30, 295(1): 119-32; Peret, T. C. et al. “Characterization of human metapneumoviruses isolated from patients in North America,” J Infect Dis 2002 Jun. 1, 185(11):1660-3; van den Hoogen, B. G. et al. “A newly discovered human pneumovirus isolated from young children with respiratory tract disease,” Nat Med 2001 Jun., 7(6):719-24). Other members of this virus family include historically significant human pathogens such as measles virus, mumps virus, parainfluenza virus, and respiratory syncytial virus. Prior to the identification of SARS, human MPV was generally associated with mild respiratory infection in humans, except for a small number of cases in individuals with serious pulmonary or immunological compromise such as leukemia. The molecular sequence of the nucleic acid genome of human MPV has recently been determined, confirming the similarity of its genome sequence to other Paramyxoviruses and indicating that human MPV is distantly related to other Paramyxovirus agents of human disease such as measles virus, mumps virus, parainfluenza virus, and respiratory syncytial virus. The molecular sequence of human MPV, which is hereby incorporated by reference in its entirety, can be accessed at the National Center for Biotechnology Information's (NCBI) web site at http://www.ncbi.nlm.nih.gov/ as Genbank reference sequence AY145301. Human MPV appears to be most similar at the molecular level to avian metapneumovirus, perhaps reflecting an introduction of the virus into the human population from an avian source at some undetermined time in the past (Njenga, M. K. et al. Metapneumoviruses in birds and humans. Virus Res. 2003 February;91(2):163-9). While there is extensive literature concerning the molecular and cell biology of Paramyxoviruses generally because of their overall significance in human disease, there is relatively little known specifically concerning human MPV. The antiviral drug ribavirin has been used to treat severe cases of human respiratory syncytial virus, which is distantly related to human MPV, and there is experimental evidence in mice that anti-inflammatory cytokines may augment ribavirin therapy (Bonville, et al., 2003 “Altered Pathogenesis of Severe Pneumovirus Infection in Response to Combined Antiviral and Specific Immunomodulatory Agents,” J. Virol. 77:1237-1244, which is hereby incorporated by reference herein in its entirety), but there is no evidence that such a therapeutic regimen is effective against SARS or human MPV infection.

Human coronavirus (human CoV) is a member of the Coronaviridae family of viruses. Various strains of human CoV have been isolated from mild outbreaks of human respiratory infection for many years, and these viruses are generally known to be a part of the diverse group of “common cold” viruses. There has been little direct characterization of human CoV and the specific aspects of its molecular and cell biology. However, there has been written a significant amount of literature regarding a murine coronvirus known as mouse hepatitis virus (MHV), which is much more severe in the mouse than human CoV has been heretofore in humans (see Luo et al., 1999, “Amino Acid Substitutions within the Leucine Zipper Domain of the Murine Coronavirus Spike Protein Cause Defects in Oligomerization and the Ability to Induce Cell-to-Cell Fusion,” J. Virol. 73: 8152-8159, which is incorporated herein by reference in its entirety). The entire molecular genome sequence of the human CoV strain involved in SARS has been determined both in Canada and at the Centers for Disease Control and Prevention (CDCP), where this new strain of human CoV was recently isolated. The genome sequence has been made available on the Internet at http://www.ncbi.nlm.nih.gov/ in advance of publication as sequence number NC_{—}004718 by the NCBI, which genome sequence is incorporated herein by reference in its entirety. Preliminary analysis of a conserved region of the genome indicates that this strain constitutes a new group within the Coronaviridae family, not closely related to any previously identified strain of the virus (Marra, M. A. et al. The Genome sequence of the SARS-associated coronavirus Science 300 (5624), 1399-1404 (2003); Rota, P. A. et al. Characterization of a novel coronavirus associated with severe acute respiratory syndrome. Science. 2003 May 30;300 (5624):1394-9. Epub 2003 May 1, each of which is hereby incorporated by reference herein in its entirety).

Human MPV and human CoV are thus very different viruses, both from one another and from other human viral pathogens. Nevertheless, drawing from our knowledge of the viruses' families, we have identified common features that may be used to design antiviral drugs effective at inhibiting infection at the cellular level.

Human MPV and human CoV are both members of a subset of all viruses known as “enveloped” viruses. Their outer layer is composed of a membranous envelope which is derived from the cellular membranes of host cells during infection. The envelope is studded with proteins encoded by the viral genome. These proteins are modified by the addition of sugar side groups at specific positions in the linear sequence of amino acids that comprise the protein and are thus termed “viral membrane glycoproteins.” Such viral membrane glycoproteins are quite variable and individual in their amino acid sequences (even sometimes from strain to strain of the same virus) and may serve a variety of functions in infection. Some of these viral membrane glycoproteins are directly anchored to the membrane because part of the protein spans the membrane—they are generally known as “viral transmembrane glycoproteins” or sometimes “spike” glycoproteins because of their shape. Other viral membrane glycoproteins, termed “viral peripheral glycoproteins,” are indirectly anchored to the viral membrane by specific association with such viral transmembrane glycoproteins, even though they do not themselves have a membrane anchor sequence. It has been discovered that a number of subcategories of viral membrane glycoproteins have general features that may be exploited for the development of specific antiviral drugs.

One subcategory includes viral membrane glycoproteins responsible for the entry of the virus into the host cell via specific binding to the host cell followed by fusion of the viral membrane with a host cell membrane, either the plasma membrane or an internal membrane (see White, J. M., 1992, “Membrane Fusion,” Science 258:917-924, which is hereby incorporated by reference herein in its entirety). The binding and fusion functions are performed by separate regions of the glycoprotein complex. Attachment is usually mediated by a viral peripheral glycoprotein, and membrane fusion or entry, is usually mediated by a viral transmembrane glycoprotein (those viral transmembrane glycoproteins that mediate fusion are known as “fusion glycoproteins” or “transmembrane fusion glycoproteins”). In many cases, viral glycoproteins responsible for binding and fusion are made together as one complex, which is later divided by a polypeptide cleavage event into two functional subunits; this happens with influenza and HIV, for instance. In other cases, such as measles, the binding and fusion functions are always separated on two different glycoproteins.

Work over the last 25 years has shown that dissimilar virus families share a similar molecular machinery and mechanism of viral entry (see FIG. 1). This similarity was first detailed by the structural studies of the viral membrane glycoprotein of influenza virus, known as the hemagglutinin (Wilson, I. A., et al. 1981. “Structure of the hemagglutinin membrane glycoprotein of influenza virus at 3 A resolution.” Nature 289: 366-373, which is hereby incorporated by reference herein in its entirety). High resolution x-ray crystallography allowed visualization of the globular head group of the hemagglutinin, which binds to the cell receptor for influenza, and the fibrous leg region of the protein complex, which anchors the protein complex to the viral membrane via a transmembrane spanning domain and induces fusion of the viral and cellular membranes. As noted, these two functional regions are activated by the proteolytic cleavage of a hemagglutinin precursor into two glycoprotein subunits that correspond to each functional region—the receptor binding glycoprotein is known as HA1 and the fusion glycoprotein is known as HA2. It was recognized in influenza virus (and measles virus) that the new amino terminus of the fusion gylycoprotein generated by this cleavage event was highly hydrophobic and of conserved sequence (White, J. M., “Membrane Fusion.” Science, vol. 258 (Nov. 6, 1992), pp. 917-924; Eckert D. M., and Kim P. S., “Mechanisms of viral membrane fusion and its inhibition.” Annu Rev Biochem. 2001, 70:777-810, each of which is hereby incorporated by reference herein in its entirety). This hydrophobic segment of amino acids is thought to be a critical functional element in the viral fusion transmembrane glycoprotein; it is thought to interact with and insert into the target membrane, inducing membrane perturbation and thereby membrane fusion. This segment of amino acids, first identified in measles virus by Choppin's lab in the early 1980s, and immediately found also in influenza virus, became known as the “fusion peptide.” The hypothesis that the fusion peptide is a critical element in fusion became known as the “fusion peptide hypothesis.”

Such structural studies converged with early efforts to use peptides as antivirals in controlling infection. Much earlier, Parke-Davis researchers had tested a series of random small peptides against a variety of viral infections and discovered that a carbobenzoxy derivative of phenylalanine-phenylalanine-glycine (z-FFG) was effective against measles virus (Miller, F. A., et al. (1968), “Antiviral activity of carbobenzoxy di- and tripeptides on Measles virus,” Applied Microbiology 16: 1489-1496; Nicolaides, E., et al. 1968 “Potential antiviral agents. Carbobenzoxy di- and tri-peptides active against Measles and herpes viruses,” Journal of Medicinal Chemistry 11: 74-79, each of which is hereby incorporated by reference herein in its entirety). These results were confirmed by more standard virological techniques in 1971 (Norrby, E. 1971, “The effect of a carbobenzoxy tripeptides on the biological activities of measles virus,” Virology 44: 599-608, which is hereby incorporated by reference herein in its entirety). Subsequent structural studies showed that z-FFG was a peptide analogue of the fusion peptide sequences at the amino termini of the measles virus and influenza virus fusion glycoproteins (Richardson, C. D., et al. (1980) “Specific inhibition of Paramyxovirus and myxovirus replication by oligopeptides and amino acid sequences similar to those at the N-termini of the F1 or HA2 viral polypeptides,” Virology 105: 205-222; Hsu, M. C. et al. (1981) “Activation of the Sendai virus Fusion protein (F) involves a conformational change with exposure of a new amino terminus,” Virology 104: 294-302; Richardson, C. D., and Choppin, P. W. (1983) “Oligopeptides that specifically inhibit membrane fusion by paramyxoviruses: studies on the site of action,” Virology 131: 518-532, each of which is hereby incorporated by reference herein in its entirety). However, the highly hydrophobic nature of the peptide and the existence of potent vaccines for each of these viruses precluded the development of z-FFG and similar peptides as clinically useful antiviral drugs.

Beginning in 1987, Gallaher and co-workers extended these studies to human immuno-deficiency virus type 1 (HIV-1), providing the first evidence that the structure of the influenza virus fusion glycoprotein and, more generally, the fusion peptide hypothesis could be extended to a superfamily of viral entry glycoproteins that crossed the lines delineating a number of otherwise dissimilar virus families. The tandem repeat of a fusion peptide motif (FLGFLG [SEQ ID NO: 28]) was located in the amino terminus of the transmembrane fusion glycoprotein subunit of HIV-1 known as gp41 (Gallaher, W. R. (1987) “Detection of a fusion peptide sequence in the transmembrane protein of human immunodeficiency virus,” Cell 50: 327-328, which is hereby incorporated by reference herein in its entirety). It was found that: “First, the gp41 transmembrane protein is likely to be the fusion glycoprotein of HIV and may be responsible for infection of cells as well as for the cytopathic effects of fusion and cytolysis. Second, as in the case of paramyxoviruses, small peptides such as Phe-Leu-Gly, its derivatives, or drugs targeted to this peptide region, may have direct inhibitory effects on HIV infection and cytopathlogy with high specific activity.” These findings were embodied in U.S. Pat. No. 4,880,779 to Gallaher, which is hereby incorporated by reference herein in its entirety, and confirmed by genetic studies of HIV (Kowalski, M., et al. 1987 “Functional Regions of the envelope glycoprotein of human immunodeficiency virus Type 1”. Science 237: 1351-1355, which is hereby incorporated by reference herein in its entirety).

By mid-1988, Gallaher and co-workers determined that the remaining structure of gp41 could be fitted to approximate the scaffold of the structure of the fusion glycoprotein of influenza virus (see Gallaher, W. R., et al. (1989) “A general model for the transmembrane proteins of HIV and other retroviruses,” AIDS Research and Human Retroviruses 5: 431-440, which is hereby incorporated by reference in its entirety). The structure of the transmembrane fusion glycoproteins of a number of viruses in the Retrovirus family were found to have similar overall structures to the transmembrane fusion glycoprotein of influenza virus, in spite of wide amino acid sequence variation. This overall structure was found to define a transmembrane fusion glycoprotein superfamily containing at its core a “coiled coil” structure. The elements of the overall structure were identified as an amino terminal fusion peptide region, followed by an extended “sided” helix termed “amphipathic” (or “N-helix,” which is hydrophobic on one side, hydrophilic on the other), a disulfide cross-linked central region, and a “charged pre-insertion helix” (or “C-helix”) just prior to membrane insertion (see FIG. 2) (Gallaher, W. R., et al. (1989) “A general model for the transmembrane proteins of HIV and other retroviruses,” AIDS Research and Human Retroviruses 5: 431-440, which is hereby incorporated by reference herein in its entirety). The helices were designated N-helix and C-helix, depending on which end of the fusion glycoprotein, N-terminus or C-terminus, it is closer to relative to the other helix. The two antiparallel helices partly wrap around two other pairs in a trimeric structure to form the coiled coil. This superfamily of viral fusion glycoproteins has come to be known as the “class I” superfamily of fusion glycoproteins.

In 1989, Gallaher extended the concept of utilizing peptide analogues of the sequence of gp41 to include analogues of the two major helical regions of HIV-1 and described this approach in a series of grant applications to the National Institutes of Health from 1989 through 1990. The applications were not funded, and, thus, the extended study of inhibitory peptides was deferred indefinitely.

In 1990, Delwart introduced the term “leucine zipper-like” to describe the helical regions in gp41 (Delwart, E. L. et al., 1990 “Retroviral envelope glycoproteins contain a ‘leucine zipper’-like repeat,” AIDS Research and Human Retroviruses 6:703-706, which is hereby incorporated by reference herein in its entirety). Although not entirely accurate, this characterization has since been widely applied to helical structural elements in viral transmembrane fusion glycoproteins. Also in 1990, Pringle's laboratory discovered that helical structural motifs could also be found in the transmembrane fusion glycoproteins of members of the Paramyxovirus family (Chambers et al., 1990, “Heptad repeat sequences are located adjacent to hydrophobic regions in several types of virus fusion glycoproteins,” J. Gen. Virology 71:3075-3080, which is hereby incorporated by reference herein in its entirety), providing additional impetus for the concept that a superfamily of viral entry proteins extended over the Orthomyxoviridae, Paramyxoviridae and Retroviridae virus families, despite significant differences in amino acid sequence and genome structure (reviewed in Gallaher, W., Henderson, L., Fermin, C., Montelaro, R., Martin, A., Qureshi, M., Ball, J., Sattentau, Q., Luo-Zhang, H., and Garry, R. (1992a). Membrane interactions of human immunodeficiency virus: Attachment, fusion and cytopathology. In “Membrane Interactions of HIV” (R. Aloia, Ed.), Vol. 6, pp. 113-142. Wiley-Liss, Inc., NY., and in Gallaher, W., Fermin, C., Henderson, L., Montelaro, R., Martin, A., Qureshi, M., Ball, J., Luo-Zhang, H., and Garry, R. (1992b). Membrane interactions of HIV: Attachment, fusion and cytopathology. Adv Membrane Fluidity 6, 113-42., each of which is hereby incorporated by reference herein in their entirety).

In 1992 and 1993, Matthews and co-workers used peptides derived from the HIV-1 gp41 sequence in an assay to determine the potential of the peptides to inhibit fusion induced by HIV-1 (Wild et al., 1992 “A synthetic peptide inhibitor of human immunodeficiency virus replication: Correlation between solution structure and viral inhibition,” Proc Natl Acad. Sci. USA. 89:10537-10541; Wild et al., 1994 “Propensity for a Leucine Zipper-Like Domain of Human Immunodeficiency Virus Type 1 gp41 to Form Oligomers Correlates With a Role in Virus-Induced Fusion Rather Than Assembly of the Glycoprotein Complex,” Proc Natl Acad. Sci USA 91:12676-30, each of which is hereby incorporated by reference herein in its entirety). These findings were embodied in U.S. Pat. No. 5,464,933 to Bolognesi, et al. and U.S. Pat. No. 5,656,480 to Wild, et al. The length and location of the inhibitory peptides, including the drug Fuzeon™ recently licensed for use against HIV by the Food and Drug Administration, was set by the length of the amphipathic helix first described by Gallaher (Rimsky et al., 1998, “Determinants of Human Immunodeficiency Virus type 1 Resistance to gp41-derived Inhibitory Peptides”, J. Virol. 72:986-993, which is hereby incorporated by reference herein in its entirety).

In 1993, Carr and Kim demonstrated that the fusion glycoprotein of influenza virus undergoes a “spring-loaded” conformational change in the course of being activated into a fusogenic form, triggering the action of the fusion peptide. (Carr, C. M. and Kim, P. S. “A spring-loaded mechanism for the conformational change of influenza hemagglutinin,” Cell. 1993 May 21;73(4):823-32, which is hereby incorporated by reference herein in its entirety). It has since been theorized that peptide inhibitors of fusion modeled after the sequence of fusion glycoproteins may function by interfering with this essential conformational change, possibly by preventing the firing of the fusion peptide towards its target cellular membrane. (Chen, et al. 1994, “Functional role of the zipper motif region of human immunodeficiency virus type 1 transmembrane protein gp41,” J. Virology 68:2002-2010, which is hereby incorporated by reference herein in its entirety).

In 1996, Lambert and co-workers extended this rationale to the Paramyxoviruses, which had been suggested five years earlier by Chambers to also contain coiled coil structures (see Chambers, et al., 1990 “Heptad repeat sequences are located adjacent to hydrophobic regions in several types of virus fusion glycoproteins,” J Gen Virology 71:3075-3080, which is hereby incorporated by reference in its entirety). Specific peptide analogues were designed of the amino acid sequences of the fusion glycoproteins for measles virus, respiratory syncytial virus, and humar parainfluenza virus (Lambert, D. M., et al. “Peptides from conserved regions of paramyxovirus fusion (F) proteins are potent inhibitors of viral fusion.” Proc Natl Acad Sci USA. 1996 Mar. 5;93(5):2186-91, which is hereby incorporated by reference herein in its entirety). These discoveries were embodied in U.S. Pat. No. 6,479,055 to Bolognesi, et al. This patent also teaches that a computer-assisted search technology may be used to identify coiled coil motifs as candidates for fusion inhibitors. The computer technology described is neither necessary nor sufficient, with a specificity so low that the tables included in the patent enumerate approximately 13,000 peptides, varying in length from 15 to hundreds of amino acids in length, the vast bulk of which are likely irrelevant to the process of membrane fusion. The peptides described in the present invention are not included even in that extensive enumeration of peptides.

In 1996 and 2001, respectively, the viral entry glycoprotein superfamily was extended by Gallaher and co-workers to Ebola virus of the Filoviridae family (Gallaher, W. R. (1996) “Similar structural models of the transmembrane proteins of Ebola and Avian sarcoma viruses,” Cell. 85: 477-478, which is hereby incorporated by reference herein in its entirety) and to Lassa fever virus of the Arenaviridae family (Gallaher, W. R., et al. “The viral transmembrane superfamily: possible divergence of Arenavirus and Filovirus glycoproteins from a common RNA virus ancestor.” BMC Microbiol. 2001;1(1):1, which is hereby incorporated by reference herein in its entirety), both of which are agents of hemorrhagic fevers (see FIG. 3) (see also U.S. Pat. No. 6,713,069 to Gallaher, which is hereby incorporated by reference herein in its entirety). Potentially inhibitor effective peptides have also been identified from the amino acid sequences of fusion glycoproteins from the Filoviridae, from other retroviruses, such as human T-cell leukemia virus (Pinon et al., 2003, “An Antiviral Peptide Targets a Coiled-Coil Domain of the Human T-Cell Leukemia Virus Envelope Glycoprotein,” J. Virol. 77:3281-3290, which is hereby incorporated by reference herein in its entirety) and from feline immunodeficiency virus (Medinas, R. J., et al. “C-Terminal gp40 peptide analogs inhibit feline immunodeficiency virus: cell fusion and virus spread.” J. Virol. 2002 September;76(18):9079-86, which is hereby incorporated by reference herein in its entirety).

Most of the fusion glycoprotein peptide analogues which have been proposed as antivirals thus far are “long” (about 30 amino acids or more). A defect of such long peptide analogues is that, for example, in the case of HIV-1 inhibition, the peptides must be administered by subcutaneous injection. More recently, Kim and co-workers have created variants of the HIV-1 peptide analogues which are intended to provide small molecules as inhibitors that may ultimately be orally administered (Sia, S. K., et al. “Short constrained peptides that inhibit HIV-1 entry.” Proc Natl Acad Sci USA. 2002 Nov. 12;99(23):14664-9; Eckert, D. M., Kim, P. S. “Design of potent inhibitors of HIV-1 entry from the gp41 N-peptide region,” Proc Natl Acad Sci USA. 2001 Sep. 25;98(20):11187-92; Root, M. J., et al. “Protein design of an HIV-1 entry inhibitor,” Science 2001 Feb. 2;291(5505):884-8; Eckert, D. M., et al. “Inhibiting HIV-1 entry: discovery of D-peptide inhibitors that target the gp41 coiled-coil pocket,” Cell 1999 Oct. 1;99(1):103-15, each of which is hereby incorporated by reference herein in its entirety). Thus far, however, these variants have not exhibited the potency of the long peptides, and they have not been tested in humans.

In sum, work in several laboratories established that there is a superfamily of viral transmembrane fusion glycoproteins, quite variable in size and amino acid sequence, which extends to a superfamily of viruses which includes Orthomyxoviruses, Paramyxoviruses, Filoviruses, Arenaviruses and Retroviruses, five virus families that differ widely in genome structure and replication strategy (see FIG. 4). By alignment of the fusion peptide and membrane-spanning regions of the corresponding fusion glycoproteins, and by examining the vicinal sequences for possible alpha helical sequence motifs, potential inhibitory effective peptides may be designed for each individual member of these virus families.

Coronaviruses have long been considered unique and very distant outliers from the viruses which contain the superfamily of fusion glycoproteins discussed above. The genome structure and replication strategy of Coronaviruses is markedly different, and the entry proteins themselves are more complex and of a different overall structure. A coiled coil model of a MHV membrane glycoprotein was presented as long ago as 1987 (deGroot et al 1987, “Evidence for a Coiled-coil Structure in the Spike Proteins of Coronaviruses” J Mol Biol 196:963-6, which is hereby incorporated by reference herein in its entirety), and the identification of the fusion glycoprotein of MHV and the identification of extended “leucine zipper” heptad repeat motifs (FIG. 5) was achieved by mutational analyses (Luo et al., 1999 “Amino Acid Substitutions within the Leucine Zipper Domain of the Murine Coronavirus Spike Protein Cause Defects in Oligomerization and the Ability to Induce Cell-to-Cell Fusion,” J. Virol 73: 8152-8159, which is incorporated herein by reference in its entirety). Such analyses establish a latter carboxy-terminal half of the fusion glycoprotein, which is generally cleaved from the first half by an endoproteolytic enzyme, as the glycoprotein most responsible for coronavirus-induced membrane fusion. However, three factors prevented the CoV fusion glycoprotein (also known as the “S2 glycoprotein,” “S2,” or “the S2 subunit”) from being included in the superfamily of fusion glycoproteins discussed above. First, there is no canonical fusion peptide motif. Second, there is a great deal of amino acid sequence in the S2 glycoprotein prior to the first heptad repeat motif, which is unprecedented in the other virus families. Third, there is an extensive disulfide cross-linked region between the two heptad repeat motifs.

We recently examined these apparent dissimilarities in the context of the more unusual members of the virus families already included in the viral entry glycoprotein superfamily, such as the spumaretroviruses which contain large inserts of extra amino acid sequence and lack clearly defined fusion peptides. We found that the amino acids of the SARS CoV fusion glycoprotein located prior to membrane insertion can in fact be modeled as a similar structure to the viral fusion glycoproteins seen in HIV-1 and the other Retroviruses and Filoviruses, with an approximately 100 amino acid disulfide cross-linked region between the two heptad repeat regions (see FIG. 6). Also, the charged pre-insertion helix (with 16 charged amino acids out of 56 total) of the SARS CoV fusion glycoprotein is followed by a region rich in aromatic amino acids highly similar to corresponding regions in HIV-1 and Ebola virus.

The peptide sequence of the fusion glycoprotein of the SARS CoV (Urbani strain AY278741) can be fitted to the Gallaher et al. (1989) general scaffold of the gp41 fusion glycoprotein (also known as “TM”) of HIV-1 (see FIG. 6). While lacking x-ray crystallographic or other biophysical data needed for confirmation, this model is consistent with the proven structures of other viral fusion glycoproteins, beginning with the influenza virus hemagglutinin in 1981 (Wilson, I. A., et al. 1981. “Structure of the haemagglutinin Membrane glycoprotein of influenza virus at 3 A resolution,” Nature 289, 366-373, which is hereby incorporated by reference herein in its entirety), as well as with similar suggestions and experimental data in other coronavirus systems from other laboratories (e.g., see Luo, et al., 1999 “Amino Acid Substitutions within the Leucine Zipper Domain of the Murine Coronavirus Spike Protein Cause Defects in Oligomerization and the Ability to Induce Cell-to-Cell Fusion,” J Virol 73: 8152-8159; Zelus et al. (2003). Conformational changes in the spike glycoprotein of murine Coronaviruses are induced at 37 degrees C. either by soluble murine CEACAM1 receptors or by pH 8. J Virol 77, 830-40, each of which is hereby incorporated by reference herein in its entirety). While cartoon models of the CoV fusion glycoprotein as a coiled coil were proposed as early as 1987 (de Groot et al. (1987). Sequence and structure of the coronavirus peplomer protein. Adv Exp Med Biol 218, 31-8, which is hereby incorporated by reference herein in its entirety), previous models have not been presented in this detail or demonstrated such close parallels with the other fusion glycoproteins. The detailed model presented here (FIG. 7), shown in comparison to the known features and structure of HIV-1 TM glycoprotein, has significant implications for avenues to develop antiviral drugs that function as fusion inhibitors of the SARS CoV.

First, there is a minimum furin-like cleavage site located at amino acids 758-762. Beginning about amino acid 900 there is an extended heptad repeat region similar to the N-helix of the HIV-1 transmembrane glycoprotein. This region differs from the N-helices of the fusion glycoproteins of retroviruses, filoviruses (Ebola virus) and arenaviruses (Lassa fever virus) principally in the extraordinarily length of the helix (see Gallaher, W. R. 1996 “Similar structural models of the transmembrane proteins of Ebola and Avian sarcoma viruses,” Cell 85: 477-478; Gallaher, W. R., et al. 1989 “A general model for the transmembrane proteins of HIV and other retroviruses,” AIDS Research and Human Retroviruses 5, 431-440; Gallaher et al. (2001). The viral transmembrane superfamily: possible divergence of arenavirus and filovirus glycoproteins from a common RNA virus ancestor. BMC Microbiol 1, 1, each of which is incorporated by reference herein in its entirety) (see FIG. 8). While there are helix-breaking motifs present (e.g., TTTS [SEQ ID NO: 29]), the helix may be stabilized in such areas by the very strong heptad repeat of hydrophobic amino acids along the left side of the helix projection. At 17 nm, this helix is overly long for the known dimensions of the coronavirus surface spike, but may reflect an extension that occurs upon binding or configurational alteration of the protein while in the process of becoming a fusion-active form.

Second, there is a short region bounded by cysteines, (see FIG. 7) which is so similar to that of the fusion glycoproteins of the retroviruses and Ebola virus to prompt us to model it as a similar disulfide-stabilized apex.

Third, there is a region with several sites (shown by stick figures in FIG. 7) for possible N-linked glycosylation that, like HIV-1, are only found after the disulfide-linked apex. This region is highly variable among Coronavirus membrane glycoproteins proteins, not unlike the variability among the retrovirus transmembrane glycoproteins.

Fourth, there is a region prior to the point the SARS CoV fusion glycoprotein is anchored in the viral envelope membrane, which has a high percentage of charged amino acids, a strong propensity to form an α helix, and a heptad repeat, so that it is comparable to the C-helix (known as HR2) of the HIV-1 transmembrane glycoprotein. SARS CoV and other CoV have well-conserved “leucine-zipper-like” motifs in the C-helix with leucine or isoleucines spaced such that they would form a highly hydrophobic face along the helix (Luo et al. (1999). Amino Acid Substitutions within the Leucine Zipper Domain of the Murine Coronavirus Spike Protein Cause Defects in Oligomerization and the Ability to Induce Cell-to-Cell Fusion. J. Virol. 73: 8152-8159, which is hereby incorporated by reference herein in its entirety). It has been demonstrated that mutations in this region of the C-helix of the MHV fusion glycorprotein cause defects in oligomerization and the ability to induce cell:cell fusion. (Luo et al. (1999). Amino Acid Substitutions within the Leucine Zipper Domain of the Murine Coronavirus Spike Protein Cause Defects in Oligomerization and the Ability to Induce Cell-to-Cell Fusion. J. Virol. 73: 8152-8159). The N-helix of the SARS CoV also has a readily identifiable “leucine-zipper-like” motif. Although the “leucine-zipper” is not as evident in the N-helices of other CoV, the N- and C-helices may nevertheless interact to form a “hydrophobic groove” or other non-covalent interactions (see Bosch, B. J., van der Zee, R., de Haan, C. A., and Rottier, P. J. (2003). The coronavirus spike protein is a class I virus fusion protein: structural and functional characterization of the fusion core complex. J Virol 77, 8801-8811, which is hereby incorporated by reference herein in its entirety). The “hydrophobic groove” is a groove or slot in the antiparallel helical structure that is lined with hydrophobic amino acids. The “leucine-zipper-like” motifs, with amino acids in the predicted hydrophobic grove of the SARS CoV fusion glycoprotein, marked by asterisks, are depicted in FIG. 7.

The amino terminal end of this charged pre-insertion helix shows a peptide motif ELDKY [SEQ ID NO: 30] highly conserved among Coronaviruses, which is very similar to a biologically significant peptide, ELDKW [SEQ ID NO: 31], in the C-helix of HIV-1 gp41. In HIV-1 this peptide is recognized as a neutralization epitope, for which a human monoclonal antibody has been developed (Muster et al. (1993). A conserved neutralizing epitope on gp41 of human immunodeficiency virus type 1. J Viol 67, 6642-7; Muster et al. (1994). Cross-neutralizing activity against divergent human immunodeficiency virus type 1 isolates induced by the gp41 sequence ELDKWAS. J Virol 68, 4031-4, each of which is hereby incorporated by reference herein in its entirety) and is in human clinical trials (Stiegler et al. (2002). Antiviral activity of the neutralizing antibodies 2F5 and 2G12 in asymptomatic HIV-1 infected humans: a phase I evaluation. AIDS 16, 2019-25, which in hereby incorporated by reference herein in its entirety) The ELDKW [SEQ ID NO: 31] motif is also represented in the recently licensed peptide fusion inhibitor, Fuzeon™, that suppresses HIV-1 infection in the nanomolar range (Kilby et al. (1998). Potent suppression of HIV-1 replication in humans by t-20, a peptide inhibitor of gp41-mediated virus entry. Nat Med 4, 1302-7, which is hereby incorporated by reference herein in its entirety).

Finally, just prior to membrane insertion (the membrane spanning domain was predicted by TMpred (http://www.ch.embnet.org/software/TMPRED_form.html). there is a region enriched in aromatic amino acids and extraordinarily highly conserved throughout the Coronaviridae. Because of its high interfacial propensity (Yau et al., 1998) it is unlikely that the tryptophan (W) rich aromatic domain is part of the transmembrane anchor in contrast to the prediction of Rota et al. (2003). This region lies in an identical location to comparable aromatic rich regions in the fusion glycoproteins of HIV-1 and Ebola virus, which have been shown to be fusogenic in liposome systems (Suarez, et al., 2000 “Membrane Interface-Interacting Sequences within the Ectodomain of the Human Immunodeficiency Virus type 1 Envelope Glycoprotein: Putative Role During Viral Fusion,” J. Virol. 74:8038-8047, which is hereby incorporated by reference herein in its entirety). An experimental octapeptide mimicking this region of the feline immunodeficiency virus (FUV) transmembrane glycoprotein has been found to inhibit fusion by that retrovirus in cell culture (Giannecchini et al., 2003 “Antiviral Activity and Conoformational Features of an Octapeptide Derived from the Membrane-Proximal Ectodomain of the Feline Immunodeficiency Virus Transmembrane Ectodomain,” J. Virol. 77:3724-3733, which is hereby incorporated by reference herein in its entirety).

We have not modeled further toward the amino terminus of the SARS CoV fusion glycoprotein, since there are no parallels established among other viruses for the structure of the fusion glycoprotein prior to the N-helix. This region, including the receptor-binding domain, is only shown schematically in FIG. 7 as a large ellipse corresponding to the large globular head group that forms the top of the characteristic “lollipop” spike seen in electron micrographs of the Coronavirus, giving it the “crown-like” appearance from which the virus family derives its name.

FIG. 9 illustrates our hypothetical mechanism for SARS CoV virion-cell fusion. PANEL A shows binding of the SARS CoV membrane glycoprotein to the cell receptor. Class I viral fusion proteins have a fusion peptide at the amino terminus, two extended α helices (N-helix and C-helix) and most have an aromatic rich domain proximal to the transmembrane anchor. Although it has been proposed that the viral entry glycoprotein of SARS CoV is not cleaved into S1 and S2 (also known as the “fusion glycoprotein”) subunits (see Rota et al., 2003), the presence of a minimal furin cleavage site suggests that the viral entry glycoprotein is cleaved. PANEL B shows rearrangement of the helical domains of the viral entry glycoprotein. The rearrangement allows the putative fusion peptide to interact with the cell plasma membrane. S1 is released from S2 in CoV when cleavage occurs. The fusion peptide may also reside between the N and C helical domains (Luo et al., 1999). PANEL C shows the helical domains of S2 “snap back” bringing the viral and cell membrane in closer proximity, and resulting in membrane deformation or “nipple” formation. Alternatively, the rearrangement of the S2 protein into the six-helix bundle confirmation does not result in nipple formation, but rather the virion itself is drawn closer to the cell surface. The fusion peptide, aromatic domain, and transmembrane anchor then constitute a contiguous track of sequences that can facilitate the flow of lipid between the two membranes. PANEL D shows the six helix bundle formation driving the cellular and viral membrane closer together resulting in spontaneous hemifusion. Peptide mimics (e.g. Fuzeon™-like peptides) of the paired helices and/or the aromatic domain are expected to block 6-helix formation in this step or in the alternative arrangement of PANEL C. PANEL E shows the fusion pore permitting cytoplasmic entry of the SARS CoV core.

The structural parallel of the helical fibrous region of the SARS CoV fusion glycoprotein to the HIV-1 transmembrane glycoprotein and other members of the same superfamily of viral transmembrane glycoproteins offers considerable support for the predicted fusion inhibitory effects of antiviral peptides modeled from the amino acid sequence of the SARS CoV fusion glycoprotein. Structural evidence has recently been provided that is consistent with this model, further suggesting that the coronavirus fusion glycoprotein is a class I fusion glycoprotein (Bosch, B. J., van der Zee, R., de Haan, C. A., and Rottier, P. J. (2003). The coronavirus spike protein is a class I virus fusion protein: structural and functional characterization of the fusion core complex. J Virol 77, 8801-8811, which is hereby incorporated by reference herein in its entirety). Furthermore, it has been demonstrated that amino acid substitutions in the N-helix (HR1) affect MHV spread in the central nervous system, and also confirmed the role of this domain in defining pH requirements for cell:cell, fusion and entry (Tsai, J. C. et al. (2003). Amino acid substitutions within the heptad repeat domain 1 of murine coronavirus spike protein restrict viral antigen spread in the central nervous system. Virology 312, 369-380, which is hereby incorporated by reference herein in its entirety). Dutch researchers have demonstrated that long synthetic peptides corresponding to the N-helix (HR1) and C-helix (HR2) of the MHV fusion glycoprotein form stable antiparallel helical complexes (Bosch, B. J. et al. (2003). The coronavirus spike protein is a class I virus fusion protein: structural and functional characterization of the fusion core complex. J Virol 77, 8801-8811, which is hereby incorporated by reference herein in its entirety). These researchers also demonstrated that a C-helix peptide could inhibit virus entry and cell:cell fusion mediated by the MHV fusion glycoprotein.

This latter study confirmed our earlier hypothesis. We had previously predicted the detailed SARS CoV S glycoprotein model (FIG. 6) that fusion inhibitory peptides may be designed from the amino acid sequence of the fusion glycoprotein of the SARS CoV by the methods disclosed herein. No such peptides had been previously proposed to inhibit coronavirus infection.

### BRIEF DESCRIPTION OF THE INVENTION

In one embodiment, the present invention relates to a method of inhibiting human metapneumoviral infection and/or human coronavirus infection which comprises administering to a host an inhibitory effective amount of a peptide or peptides comprising an inhibitory effective sequence derived from the sequence of the fusion glycoproteins of human metapneumovirus or human coronavirus, respectively. While the invention may be used in any case of human infection by these respiratory viruses, the principal target of inhibition is to prevent or reduce the severity of SARS. Reference to SARS is intended to encompass any condition meeting the case definition of SARS established by the CDCP or by the World Health Organization (WHO).

The inhibitory peptides are designed as analogues to the amino acid sequence of the metapneumovirus and coronavirus fusion glycoproteins corresponding to regions of those proteins within the linear sequence of about 100 amino acids which lie just prior to the membrane spanning sequence that anchors the glycoprotein complex to the viral membrane. In one aspect, the relevant amino acid sequences for peptides derived from metapneumovirus are:

| | |
|---|---|
| YQLSKVEGEQHVIKGRPVSSSFDPIKFPEDQFNV | [SEQ ID NO: 01] |
| ALDQVFESIENSQALVDQSNKILNSAEKGNTGF, | |

and a selection of discreet sub-sequences and derivatives thereof, as defined below. In one aspect, the relevant sequences for peptides derived from human coronavirus are:

| | |
|---|---|
| PELDSFKEELDKYFKNHTSPDVDLGDISGINASV | [SEQ ID NO: 02] |
| VNIQKEIDRLNEVAKNLNESLIDLQELGKYEQYI | |
| KWPWYVWLGF and | |
| PNLPDFKEELDQWFKNQTSVAPDLSLDYINVTFL | [SEQ ID NO: 20], |
| DLQVEMNRLQEAIKVLNQSYINLKDIGTYEYYVK | |
| WPWYVW, | |

and a selection of discreet sub-sequences and derivatives thereof, as defined below. For each sequence discussed herein, amino acids are defined by standard single letter code, defined by convention as follows:

| | | |
|---|---|---|
| A = Alanine | C = Cysteine | D = Aspartate |
| E = Glutamate | F = Phenylalanine | G = Glycine |
| H = Histidine | I = Isoleucine | K = Lysine |
| L = Leucine | M = Methionine | N = Asparagine |
| P = Proline | Q = Glutamine | R = Arginine |
| S = Serine | T = Threonine | V = Valine |
| W = Tryptophane | Y = Tyrosine | |

In each case, the peptide or peptides to be administered may be given singly or in combination, and either naturally occurring or synthetic amino acids may be used for synthetic generation of peptides, or the peptides may be generated by translation in vivo or in vitro from a DNA plasmid coding for the sequence.

The overall region from which these peptides are derived has been shown in several viral systems, including the Paramyxoviruses and Coronaviruses that are the subject of his invention, to be critical in the fusion and entry mechanisms leading to infection of human cells.

### BRIEF DESCRIPTION OF THE DRAWINGS

FIG. 1 illustrates the different morphological forms of enveloped viruses and the common overall structure of the fusion machinery (i.e., the fusion peptide(s) in concert with the antiparallel N-helix and C-helix) used for cell entry, in this case for Ebola virus and HIV-1.

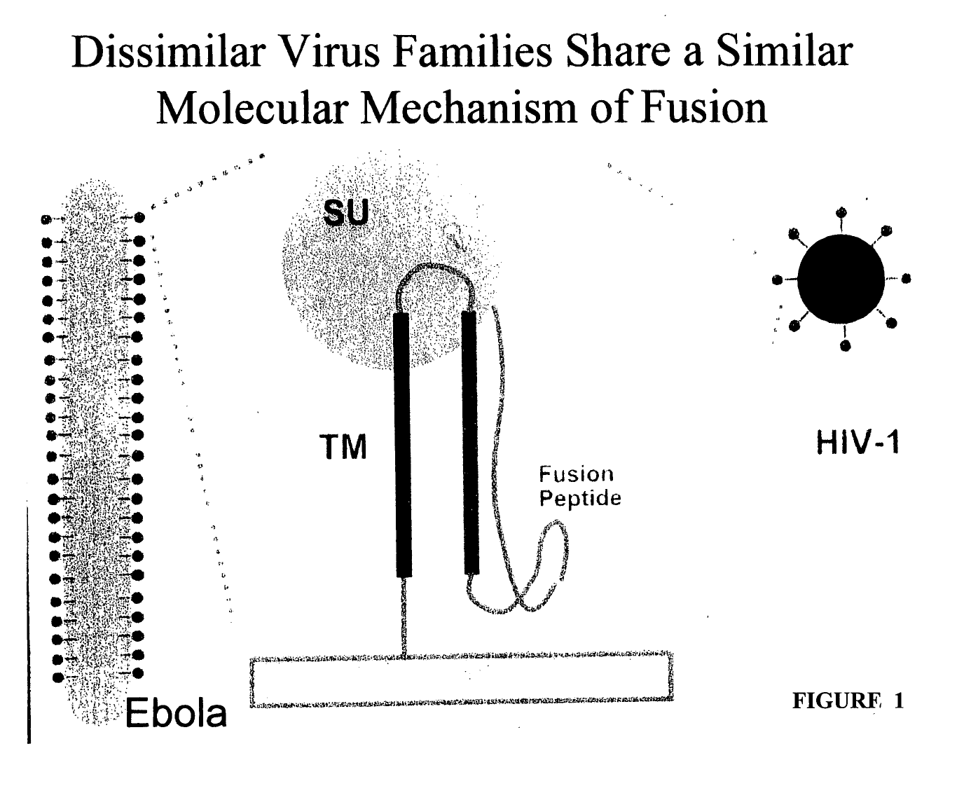

FIG. 2 illustrates the 1988 Gallaher model of the viral transmembrane fusion glycoprotein of HIV-1, gp41, which provided the basis for identifying functional helices in such proteins and the design of antiviral drugs based on those helical structures.

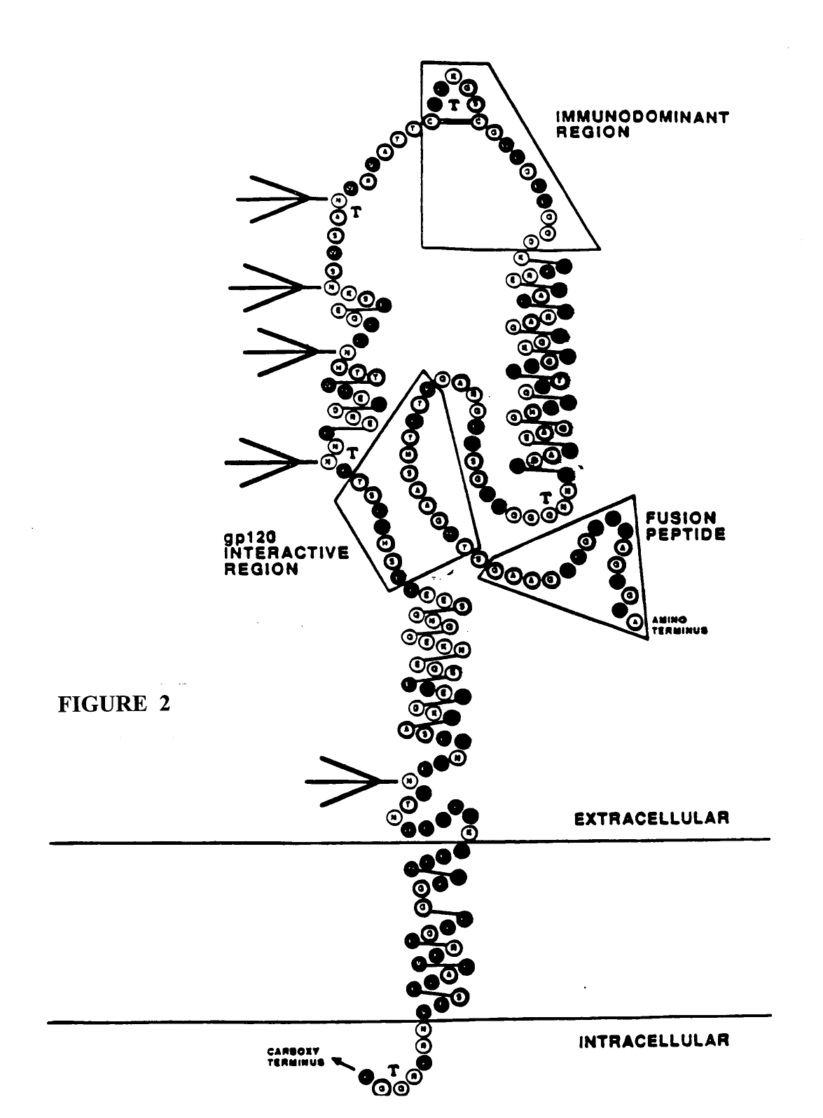

FIG. 3 illustrates the published models by Gallaher and co-workers for the fusion glycoproteins of Ebola virus and Lassa fever virus, agents of African hemorrhagic fevers, that show a striking similarity to the Gallaher model of HIV-1 gp41.

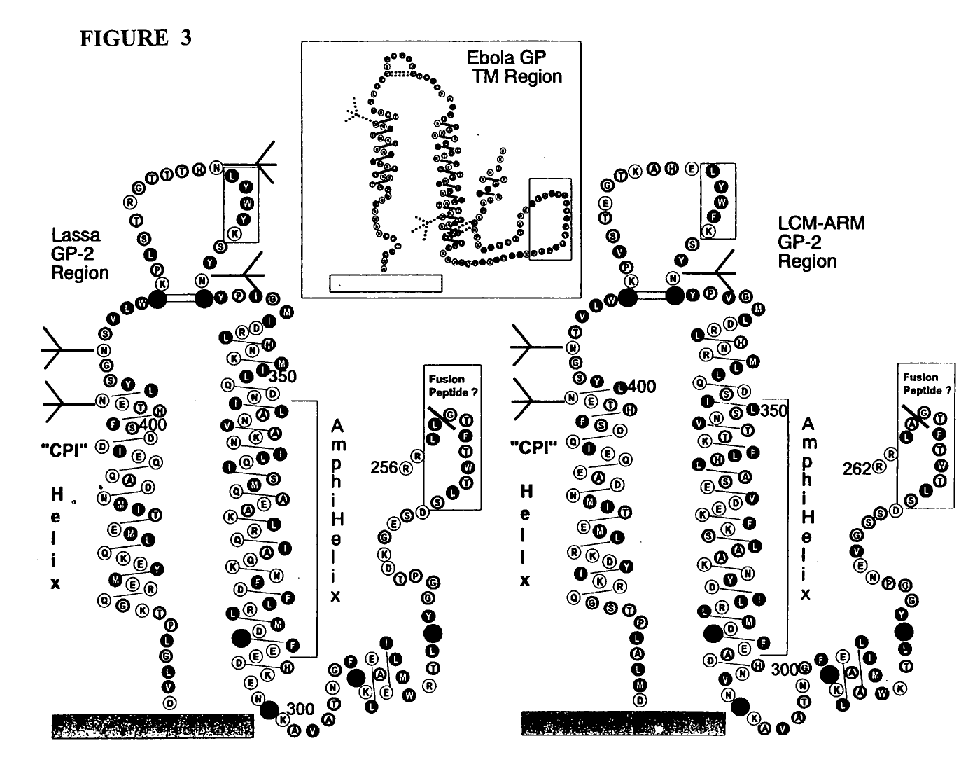

FIG. 4 shows in cartoon form the overall structural similarity of models for the viral fusion glycoproteins from five separate virus families, with significant differences in genome structure and replication strategy.

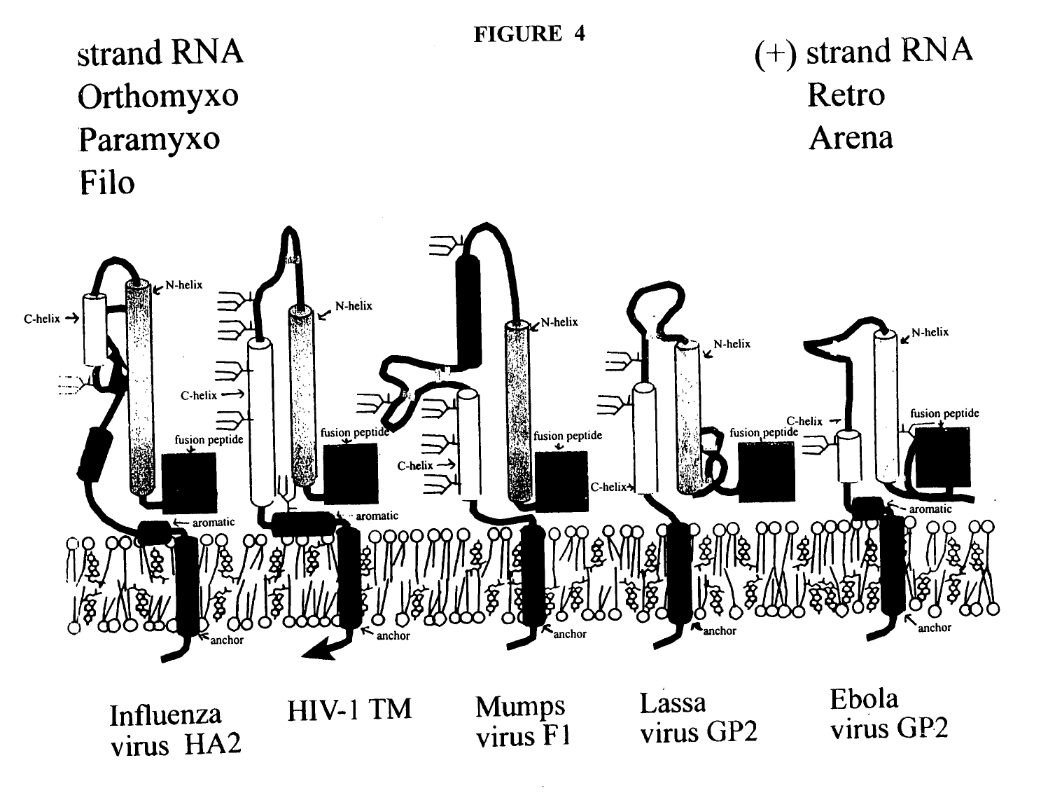

FIG. 5 shows a linear cartoon of the amino acid sequence of MHV, with the heptad repeats (HR1 and HR2) and the membrane-spanning (MS) region annotated, showing the large amount of amino acid sequence both prior to the first heptad repeat and between the heptad repeats.

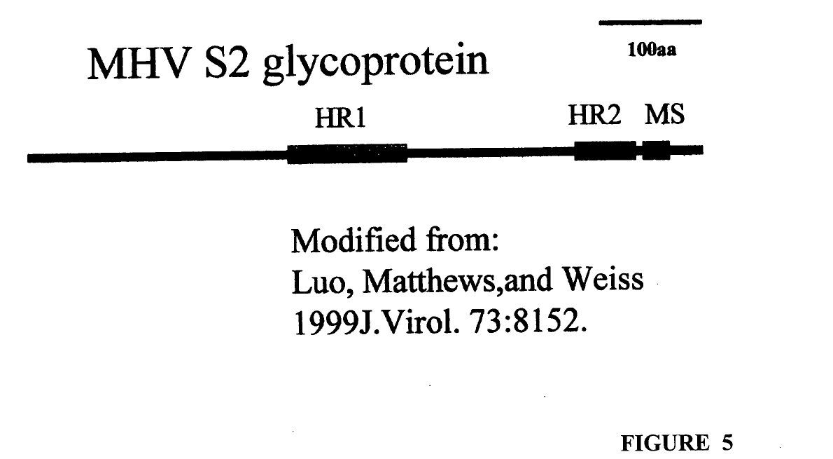

FIG. 6 illustrates a model of the SARS Coronavirus fusion glycoprotein by Garry and Gallaher illustrating the structure of the 350 amino acids prior to membrane insertion, and showing the commonality of structure with the other members of the superfamily of viral entry glycoproteins.

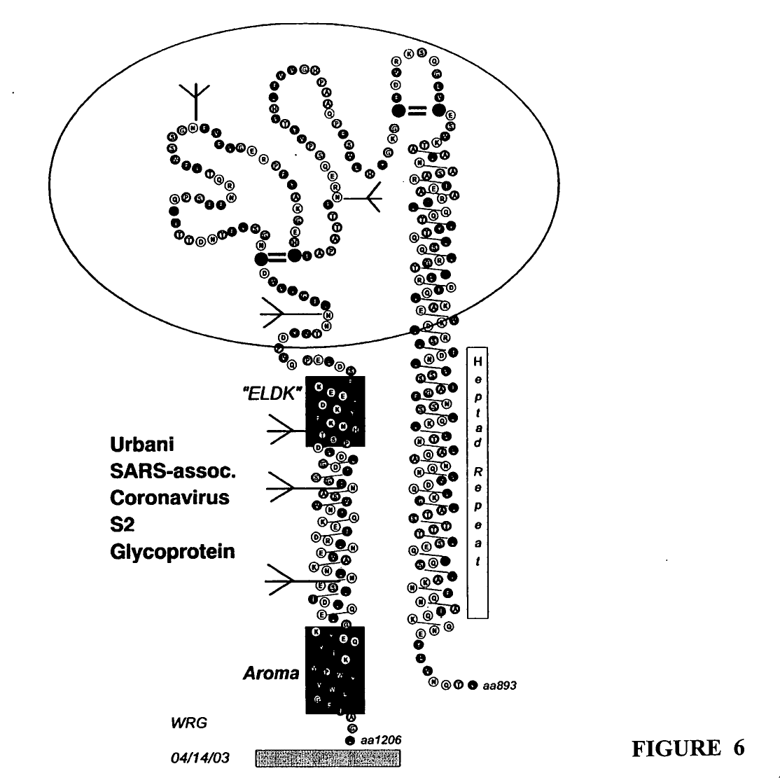

FIG. 7 shows a comparison of HIV-1 TM with SARS CoV fusion glycoprotein. At the left of FIG. 7 is an updated model of HIV-1 TM from Gallaher et al. (1989). At the right of FIG. 7 is our hypothetical model of the SARS CoV fusion glycoprotein showing motifs shared with HIV-1 TM.

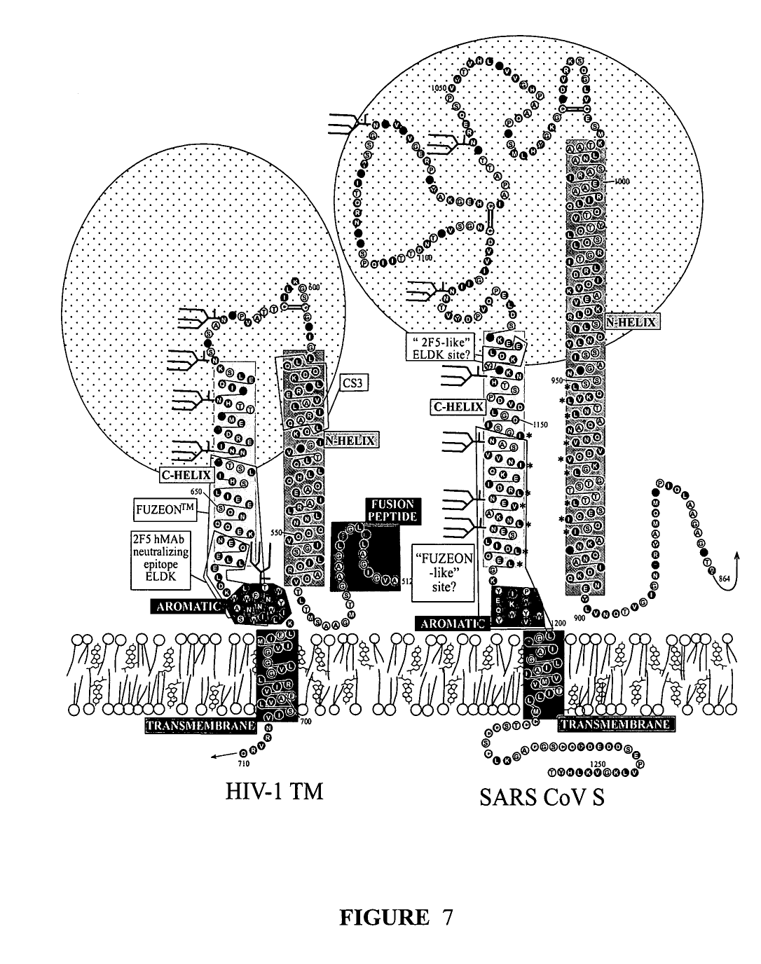

FIG. 8 illustrates the common structural features of RNA virus fusion glycoproteins. Similar motifs found in representatives of diverse virus families are depicted in order from amino terminus to carboxyl terminus. These models are based on Gallaher (1987), Gallaher et al. (2001), Gallaher et al. (1989), other references noted in the text, and our preliminary experimental results. Truncations: HIV TM C-term; measles virus F1 after N-helix; SARS CoV S N-term.

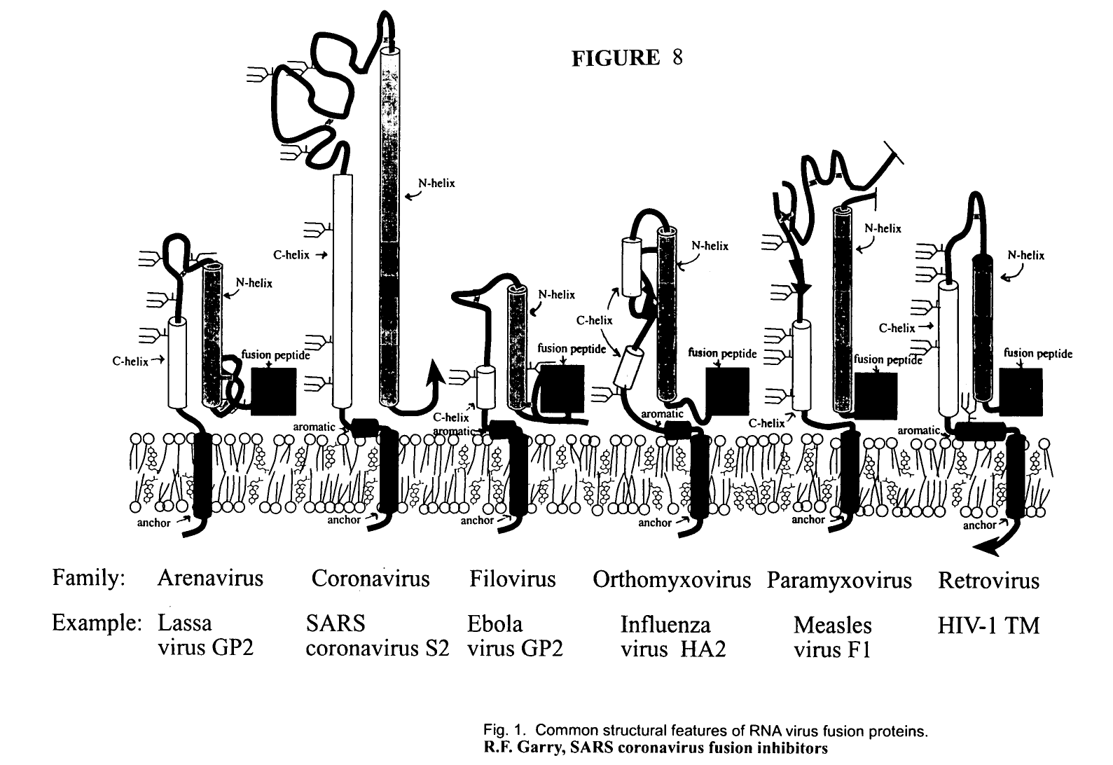

FIG. 9 illustrates our hypothetical mechanism for SARS CoV virion-cell fusion. PANEL A shows binding of the SARS CoV membrane glycoprotein to the cell receptor. Class I viral fusion proteins have a fusion peptide at the amino terminus, two extended a helices (N-helix and C-helix) and most have an aromatic rich domain proximal to the transmembrane anchor. Although it has been proposed that the viral entry glycoprotein of SARS CoV is not cleaved into S1 and S2 (also known as the “fusion glycoprotein”) subunits (see Rota et al., 2003), the presence of a minimal furin cleavage site suggests that the viral entry glycoprotein is cleaved. PANEL B shows rearrangement of the helical domains of the viral entry glycoprotein. The rearrangement allows the putative fusion peptide to interact with the cell plasma membrane. S1 is released from S2 in CoV when cleavage occurs. The fusion peptide may also reside between the N and C helical domains (Luo et al., 1999). PANEL C shows the helical domains of S2 “snap back” bringing the viral and cell membrane in closer proximity, and resulting in membrane deformation or “nipple” foration. Alternatively, the rearrangement of the S2 protein into the six-helix bundle confirmation does not result in nipple formation, but rather the virion itself is drawn closer to the cell surface. The fusion peptide, aromatic domain, and transmembrane anchor then constitute a contiguous track of sequences that can facilitate the flow of lipid between the two membranes. PANEL D shows the six helix bundle formation driving the cellular and viral membrane closer together resulting in spontaneous hemifusion. Peptide mimics (e.g. Fuzeon™-like peptides) of the paired helices and/or the aromatic domain are expected to block 6-helix formation in this step or in the alternative arrangement of PANEL C. PANEL E shows the fusion pore permiting cytoplasmic entry of the SARS CoV core.

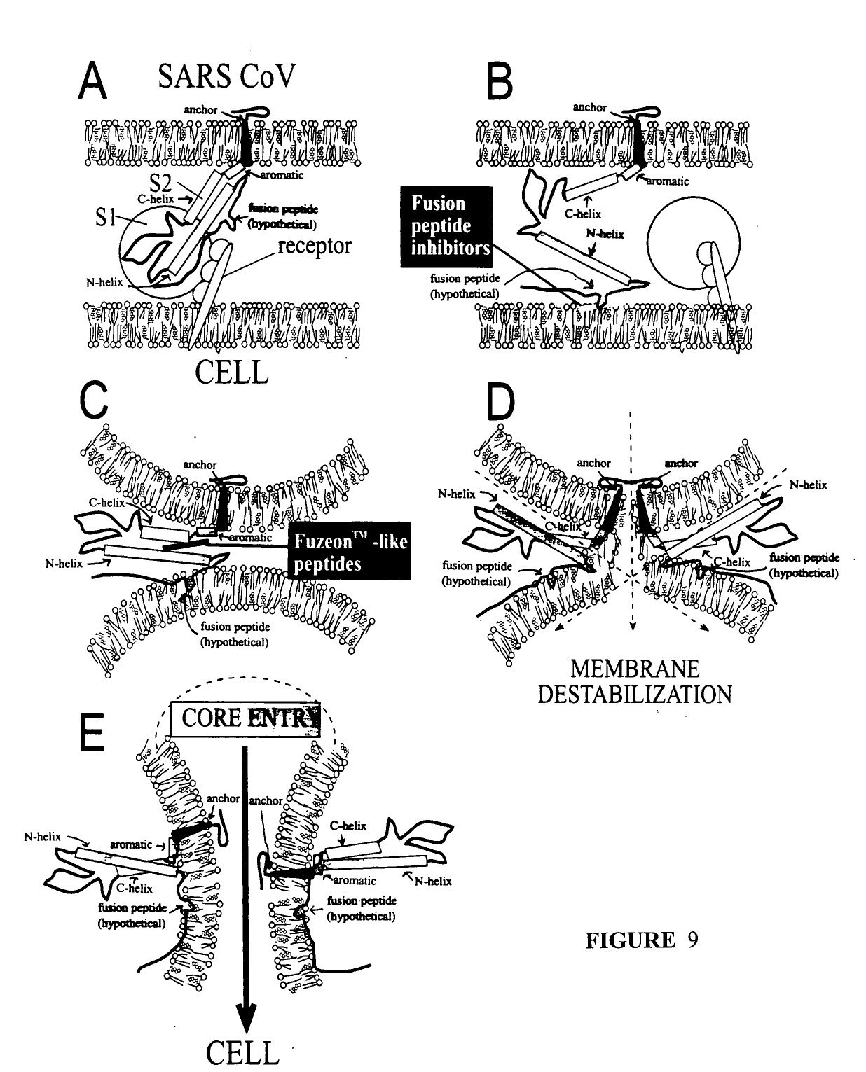

FIG. 10 contains a comparison of the amino acid sequences of the CPI helices of human coronavirus OC43, MHV A59, and SARS CoV.

FIG. 11 is a listing of peptide analogues of the CPI helix of human MPV which are predicted to be inhibitory effective.

FIG. 12 is a listing of peptide analogues of the CPI helix of SARS CoV which are predicted to be inhibitory effective.

FIG. 13 is a listing of peptide analogues of OC43 corresponding to peptide analogues of human SARS CoV; the figure also illustrates the relationship of those analogues to SEQ ID NO: 20.

FIG. 14 illustrates the results of a MHV plaque reduction assay. Approximately 70 PFU of MHV were added to monolayers of L2 target cells in duplicate wells. The upper wells are controls exposed to vehicle and the lower wells exposed to MHV pretreated with a peptide having the amino acid sequence in SEQ ID NO: 52 at a nominal concentration of 251 m. Plaques were visualized after 3 days by staining cells with crystal violet.

FIG. 15 illustrates the results of Circular dichroism (CD) spectroscopy used to delineate the structural properties of a peptide corresponding to a region of the S2 protein of MHV encompassing a portion of the C-helix and the aromatic domain (SEQ ID NO: 52). The Results show that this peptide has a domain or domains with the propensity to form an α-helix.

FIG. 16 illustrates interfacial hydrophobicity plots corresponding to sequences of SARS CoV S2, HIV-1 gp41, and EboV GP2. Interfacial hydrophobicity plots (mean values for a window of 13 residues) were generated using the Wimley and White (WW) interfacial hydrophobicity scales for individual residues (Wimley, W. C., and White, S. H. (1996) Nat Struct Biol 3, 842-848) of (PANEL A) SARS CoV strain Urbani S2 subunit (amino acids 850-1255), (PAENEL B) HIV-1 strain HXB2 gp41 (amino acids 502-710), and (PANEL C) Ebola virus strain Zaire GP2 (amino acids 520-676).

FIG. 17 shows the amino acid sequences and WW hydropathy scores of the CoV aromatic peptides. The SARS aromatic (SARS_{Aro}), MHV aromatic (MHV_{Aro}) and OC43 aromatic (OC43_{Aro}) were synthesized based on their amino acid sequence determined from GenBank accession no. AY278741 (SARS-COV strain Urbani), AY497331 (MHV strain A59), and NP_{—}937950 (Human CoV OC43). The SARS_{Aro} sequence was arbitrarily scrambled to generate the peptide SARS_{Scr}. Amino acid differences among the three CoV aromatic peptides are shown in bold and underlined text. Hydropathy scores were determined according to the Wimley and White (WW) interfacial hydrophobicity scale using a window of 13 residues.

FIG. 18 illustrates the SARS_{Aro} peptide partitions into membranes of LUV. Change in tryptophan fluorescence of SARS_{Aro} peptide as a function of increasing concentrations of LUV composed of (closed square) POPC, (closed circle) POPC:PI (9:1), (closed triangle) POPC:POPG (9:1) or (open circle) POPC:PI:CHOL (6.5:1:2.5). LUV were titrated at concentrations of 100, 250, 500, 750 and 1000 μM lipid with 2.5 M peptide. Tryptophan fluorescence values at each lipid titration (F) were normalized to tryptophan fluorescence values in potassium phosphate buffer alone (Fₒ).

FIG. 19 illustrates the results of the Tb3+/DPA microwell assays, showing that the SARS_{Aro} peptide induces leakage of LUV. Each well contained 250 μl of 50 μM DPA and 500 μM Tb3+-entrapped LUV composed of (a) POPC, (b) POPC:PI (9:1), or (c) POPC:POPG (9:1). Wells were treated with SARS_{Aro} peptide at peptide:lipid molar ratios of 1:250 or 1:100 (rows 1-2), SARS_{Scr} peptide at peptide:lipid molar ratios of 1:250 or 1:100 (rows 3-4), 20 μl of DMSO (row 4), or 20 μl of Triton-X-100 (row 5). Plates were incubated for 1 h at room temperature, and membrane permeabilization was determined by visual detection of Tb3+/DPA fluorescence.

FIG. 20 illustrates the extent of leakage from ANTS-DPX LUV induced by the SARS_{Aro} and SARS_{Scr} peptides. SARS_{Aro} peptide (PANEL A) and SARS_{Scr} peptide (PANEL B) were added to LUV composed of (closed square) POPC, (closed circle) POPC:PI (9:1), (closed triangle) POPC:POPG (9:1), (open square) POPC:CHOL (7.5:2.5). (open circle) POPC:PI:CHOL (6.5:1:2.5), or (open triangle) POPC:POPG:CHOL (6.5:1:2.5) at different peptide:lipid (P:L) molar ratios. Samples were incubated at room temperature for 24 h before measuring the extent of leakage fluorometrically.

FIG. 21 shows CD spectra (mean residue ellipticity θ) of the CoV aromatic peptides for SARS_{Aro} (PANEL A), MHV_{Aro} (PANEL B), and OC43_{Aro} (PANEL C) in 10 mM potassium phosphate buffer pH 7.0 alone (closed square) or with 1 mM LUV composed of POPC:PI (9:1) (open square) at room temperature.

### REFERENCES CITED

| | | |
|---|---|---|
| 4,880,779 | November 1989 | Gallaher |
| 4,943,627 | July 1990 | Gilbert et al. |
| 5,017,688 | May 1991 | Gilbert et al. |
| 5,464,933 | November 1995 | Bolognesi et al. |
| 5,656,480 | August 1997 | Wild et al. |
| 6,479,055 | November 2002 | Bolognesi et al. |
| 6,713,069 | March 2004 | Gallaher |

### OTHER PUBLICATIONS

Arttamangkul S., Alvarez-Maubecin V., Thomas G., Williams J. T., Grandy D. K. Binding and internalization of fluorescent opioid peptide conjugates in living cells. Mol Pharmacol. 2000 December; 58(6):1570-80.

Bodansky, M., Bodansky, A., The practice of peptide synthesis (2nd edn.), Springer Verlag, Berlin (1995).

Bonville, et al., 2003 “Altered Pathogenesis of Severe Pneumovirus Infection in Response to Combined Antiviral and Specific Immunomodulatory Agents,” J. Virol. 77:1237-1244.

Bosch, B. J., van der Zee, R., de Haan, C. A., and Rottier, P. J. (2003). The coronavirus spike protein is a class I virus fusion protein: structural and functional characterization of the fusion core complex. J Virol 77, 8801-8811.

Carr and Kim, 1993 “A spring loaded mechanism for the conformational change of influenza hemagglutinin,” Cell 73:823-832.

Chambers, et al., 1990 “Heptad repeat sequences are located adjacent to hydrophobic regions in several types of virus fusion glycoproteins,” J. Gen. Virology 71:3075-3080.

Chen et al., 1994 “Functional role of the zipper motif region of human immunodeficiency virus type 1 transmembrane protein gp41,” J. Virology 68:2002-2010.

Compton S. R., Winograd D. F., Gaertner D. J. Optimization of in vitro growth conditions for enterotropic murine coronavirus strains. J Virol Methods. 1995 April; 52(3): 301-7.

de Groot et al 1987, “Evidence for a Coiled-coil Structure in the Spike Proteins of Coronaviruses” J Mol Biol 196:963-6.

de Groot, R. J., Lenstra, J. A., Luytjes, W., Niesters, H. G., Horzinek, M. C., van der Zeisjst, B. A., and Spaan, W. J. (1987). Sequence and structure of the coronavirus peplomer protein. Adv Exp Med Biol 218, 31-8.

Delwart, E. L. et al., 1990 “Retroviral envelope glycoproteins contain a ‘leucine zipper’-like repeat,” AIDS Research and Human Retroviruses 6:703-706.

Drosten, et al. “Identification of a Novel Coronavirus in Patients with Severe Acute Respiratory Syndrome,” N Engl J Med 2003 Apr. 10.

Eckert, D. M. and Kim, P. S. “Mechanisms of viral membrane fusion and its inhibition,” Annu Rev Biochem. 2001;70:777-810.

Eckert, D. M. and Kim, P. S. “Design of potent inhibitors of HIV-1 entry from the gp41 N-peptide region,” Proc Natl Acad Sci USA. 2001 Sep. 25;98(20):11187-92.

Eckert, D. M., et al. “Inhibiting HIV-1 entry: discovery of D-peptide inhibitors that target the gp41 coiled-coil pocket,” Cell 1999 Oct. 1;99(1):103-15.

Gallaher, W., Henderson, L., Fermin, C., Montelaro, R., Martin, A., Qureshi, M., Ball, J., Sattentau, Q., Luo-Zhang, H., and Garry, R. (1992a). Membrane interactions of human immunodeficiency virus: Attachment, fusion and cytopathology. In “Membrane Interactions of HIV” (R. Aloia, Ed.), Vol. 6, pp. 113-142. Wiley-Liss, Inc., NY.

Gallaher, W. R. 1987 “Detection of a fusion peptide sequence in the transmembrane protein of human immunodeficiency virus,” Cell 50, 327-328.

Gallaher, W. R. 1996 “Similar structural models of the transmembrane proteins of Ebola and Avian sarcoma viruses,” Cell 85: 477-478.

Gallaher, W. R., Ball, J. M., Garry, R. F., Griffin, M. C., and Montelaro, R. C 1989 “A general model for the transmembrane proteins of H1V and other retroviruses,” AIDS Research and Human Retroviruses 5, 431-440.

Gallaher, W. R., de Simone, C. and Buchmeier, M. (2001). The viral transmembrane superfamily: possible divergence of arenavirus and filovirus glycoproteins from a common RNA virus ancestor. BMC Microbiol 1, 1.

Gallaher, W., Fermin, C., Henderson, L., Montelaro, R., Martin, A., Qureshi, M., Ball, J., Luo-Zhang, H., and Garry, R. (1992b). Membrane interactions of HIV: Attachment, fusion and cytopathology. Adv Membrane Fluidity 6, 113-42.

Garry, R. F. and Dash, S. (2003) Proteomics computational analyses suggest that hepatitis C virus E1 and pestivirus E2 envelope glycoproteins are truncated class II fusion proteins. Virology 307, 255-65.

Giannecchini, et al., 2003 “Antiviral Activity and Conoformational Features of an Octapeptide Derived from the Membrane-Proximal Ectodomain of the Feline Immunodeficiency Virus Transmembrane Ectodomain,” J. Virol. 77:3724-3733.

Guan, Y., Peiris, J. S., Zheng, B., Poon, L. L., Chan, K. H., Zeng, F. Y., Chan, C. W., Chan, M. N., Chen, J. D., Chow, K. Y., Hon, C. C., Hui, K. H., Li, J., Li, V. Y., Wang, Y., Leung, S. W., Yuen, K. Y., and Leung, F. C. (2004). Molecular epidemiology of the novel coronavirus that causes severe acute respiratory syndrome. Lancet 363, 99-104.

Guan, Y., Zheng, B. J., He, Y. Q., Liu, X. L., Zhuang, Z. X., Cheung, C. L., Luo, S. W., Li, P. H., Zhang, L. J., Guan, Y. J., Butt, K. M., Wong, K. L., Chan, K. W., Lim, W., Shortridge, K. F., Yuen, K. Y., Peiris, J. S., and Poon, L. L. (2003). Isolation and characterization of viruses related to the SARS coronavirus from animals in southern China. Science 302, 276-278.

Gutte, B. (ed.), Peptides: Synthesis, Structure and Application, Academic Press, San Diego (1995).

Haff, R. F. (1962) Plaque formation by a mouse hepatitis virus. Virology 18, 507-508.

Hoesl C. E., Nefzi A., Ostresh J. M., Yu Y., and Houghten, R. A. Mixture-based combinatorial libraries: from peptides and peptidomimetics to small molecule acyclic and heterocyclic compounds. Methods Enzymol. 2003;369:496-517.

Hsu, M. C. et al. 1981 “Activation of the Sendai virus Fusion protein (F) involves a conformational change with exposure of a new amino terminus,” Virology 104, 294-302.

Jemmerson “Effects of Conformation, Amino Acid Sequence, and Carrier Coupling on the Immunological Recognition of Peptide and Protein Antigens” in: Zegers et al., Immunological Recognition of Peptides in Medicine and Biology (New York, CRC, 1995), pp. 213-225.

Kay, B., Winter, J., and McCafferty, J. Phage Display of Peptides and Proteins: A Laboratory Manual, Academic Press; 1st edition (Jan. 15, 1996).

Kilby, J. M., Hopkins, S., Venetta, T. M., DiMassimo, B., Cloud, G. A., Lee, J. Y., Alldredge, L., Hunter, E., Lambert, D., Bolognesi, D., Matthews, T., Johnson, M. R., Nowak, M. A., Shaw, G. M., and Saag, M. S. (1998). Potent suppression of HIV-1 replication in humans by t-20, a peptide inhibitor of gp41-mediated virus entry. Nat Med 4, 1302-7

Kowalski, M., et al. 1987 “Functional Regions of the envelope glycoprotein of human immunodeficiency virus Type 1,” Science 237, 1351-1355.

Ksiazek, et al. “A Novel Coronavirus Associated with Severe Acute Respiratory Syndrome,” N Engl J Med 2003 Apr. 10.

Lambert, D. M., et al. “Peptides from conserved regions of paramyxovirus fusion (F) proteins are potent inhibitors of viral fusion,” Proc Natl Acad Sci USA. 1996 Mar. 5;93(5): 2186-91.

Li, W., Moore, M. J., Vasilieva, N., Sui, J., Wong, S. K., and Berne, M. A., Somasundaran, M., Sullivan, J. L., Luzuriaga, K., Greenough, T. C., Choe, H., and Farzan, M. (2003). Angiotensin-converting enzyme 2 is a functional receptor for the SARS coronavirus. Nature 426, 450-454.

Liu, S., Xiao, G., Chen, Y., He, Y., Niu, J., Escalante, C. R., Xiong, H., Farmar, J., Debnath, A. K., Tien, P., and Jiang, S. (2004). Interaction between heptad repeat 1 and 2 regions in spike protein of SARS-associated coronavirus: implications for virus fusogenic mechanism and identification of fusion inhibitors. The Lancet Vol. 363, pp. 938-940.

Luo, Z., Matthews, A. M., and Weiss, S. R. (1999). Amino Acid Substitutions within the Leucine Zipper Domain of the Murine Coronavirus Spike Protein Cause Defects in Oligomerization and the Ability to Induce Cell-to-Cell Fusion. J. Virol. 73: 8152-8159.

Marra, M. A., Jones, S. J., Astell, C. R., Holt, R. A., Brooks-Wilson, A., Butterfield, Y. S., Khattra, J., Asano, J. K., Barber, S. A., Chan, S. Y., Cloutier, A., Coughlin, S. M., Freeman, D., Girn, N., Griffith, O. L., Leach, S. R., Mayo, M., McDonald, H., Montgomery, S. B., Pandoh, P. K., Petrescu, A. S., Robertson, A. G., Schein, J. E., Siddiqui, A., Smailus, D. E., Stott, J. M., Yang, G. S., Plummer, F., Andonov, A., Artsob, H., Bastien, N., Bernard, K., Booth, T. F., Bowness, D., Drebot, M., Fernando, L., Flick, R., Garbutt, M., Gray, M., Grolla, A., Jones, S., Feldmann, H., Meyers, A., Kabani, A., Li, Y., Normand, S., Stroher, U., Tipples, G. A., Tyler, S., Vogrig, R., Ward, D., Watson, B., Brunham, R. C., Krajden, M., Petric, M., Skowronski, D. M., Upton, C., and Roper, R. L. (2003). The genome sequence of the SARS associated coronavirus. Science 300, 1399-1404.

Mayer L. D., Hope M. J., Cullis P. R. Vesicles of variable sizes produced by a rapid extrusion procedure. Biochim Biophys Acta. 1986 Jun. 13;858(1): 161-8.

Medinas, R. J., et al. “C-Terminal gp40 peptide analogs inhibit feline immunodeficiency virus: cell fusion and virus spread.” J. Virol. 2002 September; 76(18): 9079-86.

Miller, F. A., et al. 1968 “Antiviral activity of carbobenzoxy di- and tripeptides on Measles virus,” Applied Microbiology 16, 1489-1496.

Muster, T., Steindl, F., Purtscher, M., Trkola, A., Klima, A., Himmler, G., Ruker, F., and Katinger, H. (1993). A conserved neutralizing epitope on gp41 of human immunodeficiency virus type 1. J Virol 67, 6642-7.

Muster, T., Guinea, R., Trkola, A., Purtscher, M., Klima, A., Steindl, F., Palese, P., and Katinger, H. (1994). Cross-neutralizing activity against divergent human immunodeficiency virus type 1 isolates induced by the gp41 sequence ELDKWAS. J Virol 68, 4031-4.

Nash, T. C. and Buchmeier M. J. (1997). Entry of mouse hepatitis virus into cells by endosomal and nonendosomal pathways. Virology 233, 1-8.

Nicolaides, E., et al. 1968. “Potential antiviral agents. Carbobenzoxy di- and tripeptides active against Measles and herpes viruses,” Journal of Medicinal Chemistry 11, 74-79.

Nieva, J. L., Nir, S., Muga, A., Goni, F. M., and Wilschut, J. (1994). Interaction of the HIV-1 fusion peptide with phospholipid vesicles: different structural requirements for fusion and leakage. Biochemistry 33, 3201-9

Njenga M. K., Lwamba H. M., and Seal B. S. Virus Res. Metapneumoviruses in birds and humans. 2003 February; 91(2): 163-9.

Norrby, E. 1971 “The effect of a carbobenzoxy tripeptides on the biological Activities of measles virus,” Virology 44, 599-608.

Peiris, J. S., Lai, S. T., Poon, L. L., Guan, Y., Yam, L. Y., Lim, W., Nicholls, J., Yee, W. K., Yan, W. W., Cheung, M. T., Cheng, V. C., Chan, K. H., Tsang, D. N., Yung, R. W., Ng, T. K., and Yuen, K. Y. (2003). Coronavirus as a possible cause of severe acute respiratory syndrome. Lancet 361, 1319-25.

Peret, T. C., et al. “Characterization of human metapneumoviruses isolated from patients in North America,” J Infect Dis. 2002 Jun. 1;185(11): 1660-3.

Pinon, et al., 2003 “An Antiviral Peptide Targets a Coiled-Coil Domain of the Human T-Cell Leukemia Virus Envelope Glycoprotein,” J. Virol. 77:3281-3290.

Poutanen, S. M., et al. “Identification of Severe Acute Respiratory Syndrome in Canada,” N Engl J Med 2003 Mar31.

Rausch J. M. and Wimley W. C. (2001). A high-throughput screen for transmembrane pore-forming peptides. Analytical Biochemistry 293:258-63.

Raussens V, Slupsky C M, Sykes B D, Ryan R O. Lipid-bound structure of an apolipoprotein E-derived peptide. J. Biol. Chem. 2003 Jul. 11;278(28): 25998-6006. Epub 2003 Apr. 22.

Richard, J. P., Melikov, K., Vives, E., Ramos, C., Verbeure, B., Gait, M. J., Chemomordik, L. V., and Lebleu, B. (2003). Cell-penetrating Peptides A Reevaluation of the Mechanism of Cellular Uptake. The Journal of Biological Chemistry Vol. 278, No. 1, Issue of Jan. 3, pp. 585-590.

Richardson, C. D. and Choppin, P. W. 1983 “Oligopeptides that specifically Inhibit membrane fusion by paramyxoviruses: studies on the site of action,” Virology 131, 518-532.

Richardson, C. D., et al. 1980 “Specific inhibition of Paramyxovirus and myxovirus replication by oligopeptides and amino acid Sequences similar to those at the N-termini of the Fl or HA2 viral polpeptides,” Virology 105, 205-222.

Rimsky, et al., 1998 “Determinants of Human Immunodeficiency Virus type 1 Resistance to gp41-derived Inhibitory Peptides,” J. Virol. 72:986-993.

Root, M. J., et al. “Protein design of an HIV-1 entry inhibitor,” Science. 2001 Feb. 2;291(5505): 884-8.

Rota P. A., Oberste M. S., Monroe S. S., Nix W. A., Campagnoli R., Icenogle J. P., Penaranda S., Bankamp B., Maher K., Chen M. H., Tong S., Tamin A., Lowe L., Frace M., DeRisi J. L., Chen Q., Wang D., Erdman D. D., Peret T. C., Burns C., Ksiazek T. G., Rollin P. E., Sanchez A., Liffick S., Holloway B., Limor J., McCaustland K., Olsen-Rasmussen M., Fouchier R., Gunther S., Osterhaus A. D., Drosten C., Pallansch M. A., Anderson L. J., Bellini W. J. Characterization of a novel coronavirus associated with severe acute respiratory syndrome. Science. 2003 May 30.

Sia, S. K., et al. “Short constrained peptides that inhibit HIV-1 entry,” Proc Natl Acad Sci USA. 2002 Nov. 12;99(23): 14664-9.

Simmons, G., Amberg, S., Rennekamp, A. J., Piefer, A., and Bates, P. (2004). Keystone Symposium on Bioterrorism and Emerging Infectious Diseases. Abstract 215, p120.

Spiegelberg H. L., et al. (1997) “DNA immunization: a novel approach to allergen-specific immunotherapy”, Allergy 52:964-70.

Stiegler, G., Armbruster, C., Vcelar, B., Stoiber, H., Kunert, R., Michael, N. L., Jagodzinski, L. L., Ammann, C., Jager, W., Jacobson, J., Vetter, N., and Katinger, H. (2002). Antiviral activity of the neutralizing antibodies 2F5 and 2G12 in asymptomatic HIV-1 infected humans: a phase I evaluation. AIDS 16, 2019-25.

Suarez, et al., 2000 “Membrane Interface-Interacting Sequences within the Ectodomain of the Human Immunodeficiency Virus type 1 Envelope Glycoprotein: Putative Role During Viral Fusion,” J. Virol. 74:8038-8047.

Tripet, B., Howard, M. W., Jobling, M., Holmes, R. K., Holmes, K. V., and Hodges, R. S. (2004). Structural Characterization of the SARS-Coronavirus Spike S Fusion Protein Core. JBC Papers in Press. Manuscript M400759200.

Tsai, J. C., de Groot, L., Pinon, J. D., Iacono, K. T., Phillips, J. J., Seo, S. H., Lavi, E., and Weiss, S. R. (2003). Amino acid substitutions within the heptad repeat domain 1 of murine coronavirus spike protein restrict viral antigen spread in the central nervous system. Virology 312, 369-380.

van den Hoogen, B. G., et al. “A newly discovered human pneumovirus isolated from young children with respiratory tract disease,” Nat Med. 2001 June; 7(6): 719-24.

van den Hoogen, B. G., et al. “Analysis of the genomic sequence of a human metapneumovirus,” Virology 2002 Mar. 30;295(1): 119-32.

Wang, Y., Ma, W. L., Song, Y. B., Xiao, W. W., Zhang, B., Huang, H., Wang, H. M., Ma, X. D., and Zheng, W. L. (2003). Gene sequence analysis of SARS-associated coronavirus by nested RT-PCR. Di Yi Jun Yi Da Xue Xue Bao 23, 421-3

White, J. M., “Membrane Fusion,” Science, vol. 258 (Nov. 6, 1992), pp. 917-924.

White, S. H., Wimley, W. C., Ladokhin, A. S., and Hristova, K. (1998) Protein folding in membranes: determining energetics of peptide-bilayer interactions. Methods Enzymol 295, 62-87.

Wild, et al. 1992 “A synthetic peptide inhibitor of human immunodeficiency virus replication: Correlation between solution structure and viral inhibition,” Proc. Natl. Acad. Sci. USA 89:10537-10541.

Wild, et al., 1994 “Propensity for a Leucine Zipper-Like Domain of Human Immunodeficiency Virus Type 1 gp41 to Form Oligomers Correlates With a Role in Virus-Induced Fusion Rather Than Assembly of the Glycoprotein Complex,” Proc. Natl. Acad. Sci. USA 91:12676-80.

Wimley, W. C., Selsted, M. E., and White, S. H. (1994). Interactions between human defensins and lipid bilayers; evidence for formation of multimeric pores. Protein Sci 3, 1362-73.

Wimley, W. C., and White, S. H. (2000a). Designing transmembrane alpha-helices that insert spontaneously. Biochemistry 39, 4432-42.

Wimley, W. C. and White, S. H. (2000b). Determining the membrane topology of peptides by fluorescence quenching. Biochemistry 39, 161-70.

Wimley, W. C., and White, S. H. (1996) Nat Struct Biol 3, 842-848.

Wilson, I. A., et al. 1981. “Structure of the haemagglutinin Membrane glycoprotein of influenza virus at 3 A resolution,” Nature 289, 366-373.

Yao, W. M., Wimley, W. C., Gaurisch, K and White S. H. (1998) The preference of tryptophan for membrane interfaces. Biochemistry. 37(42): 14713-8.

Zelus, B. D., Schickli, J. H., Blau, D. M., Weiss, S. R., and Holmes, K. V. (2003). Conformational changes in the spike glycoprotein of murine Coronaviruses are induced at 37 degrees C. either by soluble murine CEACAMI receptors or by pH 8. J Virol 77, 830-40.

Zheng, B. J., Guan, Y., Wong, K. H., Zhou, J., Wong, K. L., Young, B. W. Y., Lu, L. W., and Lee, S. S. (2004). SARS-related virus predating SARS outbreak, Hong Kong. Emerging Infectious Diseases. e-pub Jan. 16, 2004.

### DETAILED DESCRIPTION OF THE INVENTION

For convenience in the ensuing description, the following explanations of terms are adopted. However, these explanations are intended to be exemplary only. They are not intended to limit the terms as they are described or referred to throughout the specification. Rather, these explanations are meant to include any additional aspects and/or examples of the terms as described and claimed herein.

As used herein, the terms “inhibiting,” “inhibition,” “inhibitory,” and any variants thereof are to be understood as meaning (with respect to the activity of the peptides) inhibition both in a prophylactic sense (i.e., prevention of the initial transmission of the virus to an individual), as well as in the sense of preventing the infection from becoming established or ameliorating its effects once the virus has been introduced into the body.

As used herein, the term “analogue” means a peptide or peptidomimetic compound that has the same amino acid sequence as a segment of the viral membrane glycoprotein, or is designed to mimic the stereochemical shape of a portion of the viral membrane glycoprotein.

Also, in this regard, it is contemplated that the term “amino acid” as used herein refers to both naturally occurring forms, as well as synthetic forms which have been modified by the addition of side chains or other moieties to increase solubility, biological half-life or uptake and delivery to body tissues. Both D- and L-forms of all amino acids are also contemplated, in any form including their pharmacologically acceptable salts.

In one embodiment of the present invention, analogues of a portion of the fusion glycoproteins of human CoV and human MPV are employed to inhibit the normal fusion process of the viruses in vivo. In certain aspects, the portion of the fusion glycoprotein for which these analogues have been designed is the “charged pre-insertion helix” (CPI helix). The CPI helix is that portion of the fusion glycoprotein which lies within about 100 amino acids from the point at which the fusion glycoprotein is anchored within the lipid membrane of the virus and which is characterized by a high percentage of hydrophilic amino acids that may be acidic or basic in nature and that have a recognizable propensity to form an alpha helix. As discussed above, CPI helices have been shown in a number of virus systems to be involved in the induction of cell fusion, and, in some cases, analogues of those portions have been shown to inhibit fusion. The CPI helix of a virus fusion glycoprotein may be located using the following method: First, the primary amino acid sequence of the virus entry glycoprotein, toward the carboxy terminus of the virus entry glycoprotein, is examined for a uniformly hydrophobic (i.e., consisting entirely of hydrophobic amino acids, to the exclusion of hydrophilic amino acids) sequence of about 20-25 amino acids, which uniformly hydrophobic sequence has a propensity to span the lipid envelope membrane. The membrane-spanning portion has been found to be composed of more than about 60% aliphatic and aromatic amino acids in virtually all membrane spanning glycoproteins. The 100 amino acid region preceding this membrane-spanning portion is examined for charged amino acids as well as for amino acids such as glutamine (Q), glutamate (E), alanine (A), tryptophane (W), lysine (K) and leucine (L), which have a known propensity to form an alpha helix. While the core of the CPI helix is evident by finding a concentration of such amino acids as have a strong helical propensity, the beginning of the helix is found by locating a di- or tri-peptide motif that has a propensity to “nucleate” or start the helix formation. Generally, this constitutes a pair of amino acids together which each strongly favor a helix, such as glutamate (E), glutamine (Q), phenylalanine (F), lysine (K), alanine (A), or leucine (L). This is even more strongly favored when preceded by a proline (P), particularly when no more than 2 or 3 amino acids separate the P from the di- or tri-peptide motif. For example, in the CPI helix of the SARS CoV, the sequence PEL [SEQ ID NO: 32] comprises such a nucleation motif. In MHV, a comparable nucleation motif is PDFKE [SEQ ID NO: 33]. Once the CPI helix is identified, peptide analogues of the sequence of the CPI helix can be tested for their ability to inhibit virus-induced cell fusion and viral infectivity.

In one embodiment, the present invention comprises peptides which represent analogues of the CPI helix from human metapneumovirus and the CPI helix from human coronavirus. Overall, the CPI helix of each virus entry glycoprotein is between 50 and 80 amino acids in length. Synthesis and production of peptides of this length are impracticable, due to limitations in efficiency of synthesis or purity. Therefore, peptide analogues are generally limited in practice to shorter peptides over a shorter span of the glycoproteins which are effectively inhibitory at a concentration useful for human administration. This necessarily varies with each virus system and protein portion due to variations in amino acid sequence.

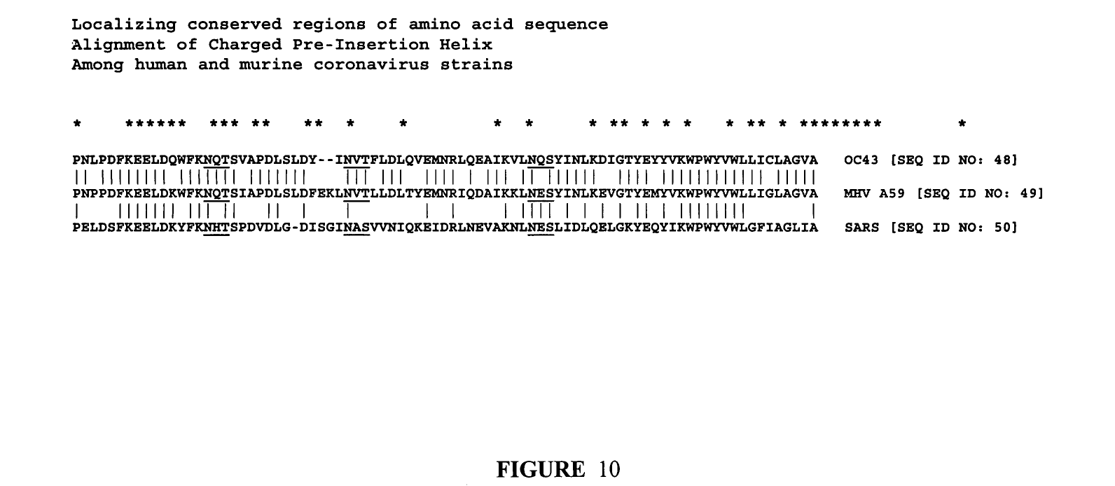

Peptides of as few as 6 amino acids or as many as 40 may provide the optimal combination of factors in development of an inhibitory peptide into a human drug. When the CPI helix has been located, it is desirable to delineate subsets of the amino acid sequence of the CPI helix which will represent inhibitory-effective peptides themselves, and together represent the best set of such peptides from the entire CPI helix. One method is to divide the entire CPI helix sequence into three segments representing about the first, second, and last third of the amino acid sequence of the CPI helix, while initiating and ending each segment with certain preferred amino acids. In general, alanine (A), glutamate (E), glutamine (Q), tyrosine (Y), phenylalanine (F), lysine (K) and proline (P) are favored as termini, and longer chain aliphatic amino acids such as valine (V), isoleucine (I) and leucine (L) are disfavored. A second, complimentary method involves centering peptides on those areas which are highly conserved in sequence among class I viral fusion glycoproteins. An example is shown in FIG. 10, which contains a comparison of the amino acid sequence of the CPI helices of human coronavirus OC43, MHV A59, and SARS CoV. Asterisks denote the identical amino acids in all three viruses, indicating a strong presumption of constancy in structure and function for those regions with a concentration of asterisks. Inhibitory effective peptides may be constructed which center on those sequences and are of decreasing lengths. Using human CoV as an example, the minimum peptide length is likely to be FKEELDK [SEQ ID NO: 34] or KWPWYVWL [SEQ ID NO: 35], the heptamer and octamer that coincide to the constant sequences at either end of the CPI helical region in HIV, MHV, and human CoV. Additional amino acids may be added to either the amino- or carboxy-termini of these conserved peptide sequences to enhance the biological and pharmacological properties of peptides used for treatment of humans using methods known to those practiced in the pharmaceutical arts. It will be apparent to those skilled in the art that other methods may be used to locate inhibitory effective peptide analogues of the amino acid CPI helix, such as screening of overlapping peptides, molecular modeling, and algorithms that utilize the Wimley-White interfacial hydrophobicity scale.

In one embodiment, inhibitory peptides are stipulated for human MPV and human CoV that range in length from 6 to 40 amino acids in length. Peptides are constructed to represent different segments of the CPI helices of these viruses that may be efficiently synthesized and inhibitory effective when used either alone or in combination.

In the case of human MPV, the CPI helix comprises the following 67 amino acids:

| | |
|---|---|
| YQLSKVEGEQHVIKGRPVSSSFDPIKFPEDQFNV | [SEQ ID NO: 01] |
| ALDQVFESIENSQALVDQSNKILNSAEKGNTGF. | |

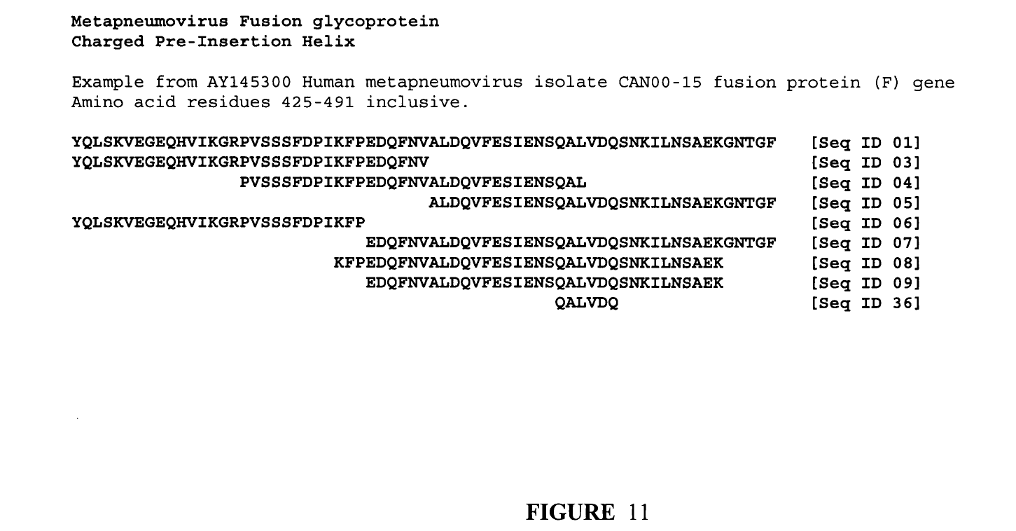

This sequence has been subdivided into 8 peptides [SEQ ID NOS: 3-9 and 36] that overlap different portions of the CPI helix amino acid sequence, as shown in FIG. 11. Any one peptide, or combination of peptides, may be used as an analogue(s) of this virus fusion glycoprotein so as to inhibit the natural interactions of this protein portion in inducing membrane fusion.

In one embodiment, the present invention comprises the following peptide analogue of the CPI helix of human MPV:

| | |
|---|---|
| EDQFNVALDQVFESIENSQA | [SEQ ID NO: 07] |
| LVDQSNKILNSAEKGNTGF. | |

This embodiment contains the maximum percentage of those amino acids, as discussed above, that define the CPI helix (i.e., Q, E, A, W, K and L), and, therefore, this analogue is predicted to be maximally active in competitively inhibiting fusion.

The minimum inhibitory effective peptide in the case of human MPV is the following hexapeptide.

| | |
|---|---|
| QALVDQ. | [SEQ ID NO: 36] |

Addition of any number of amino acids to either the amino terminus or carboxy terminus of this minimum peptide should not affect its inhibitory potential, but should have the effect of rendering the peptide more desirable for pharmaceutical use in humans.

In the case of the human SARS CoV, the CPI helix comprises the following 78 amino acid sequence:

| | |
|---|---|
| PELDSFKEELDKYFKNHTSPDVDLGDISGINASV | [SEQ ID NO: 02] |
| VNIQKEIDRLNEVAKNLNESLIDLQELGKYEQYI | |
| KWPWYVWLGF. | |

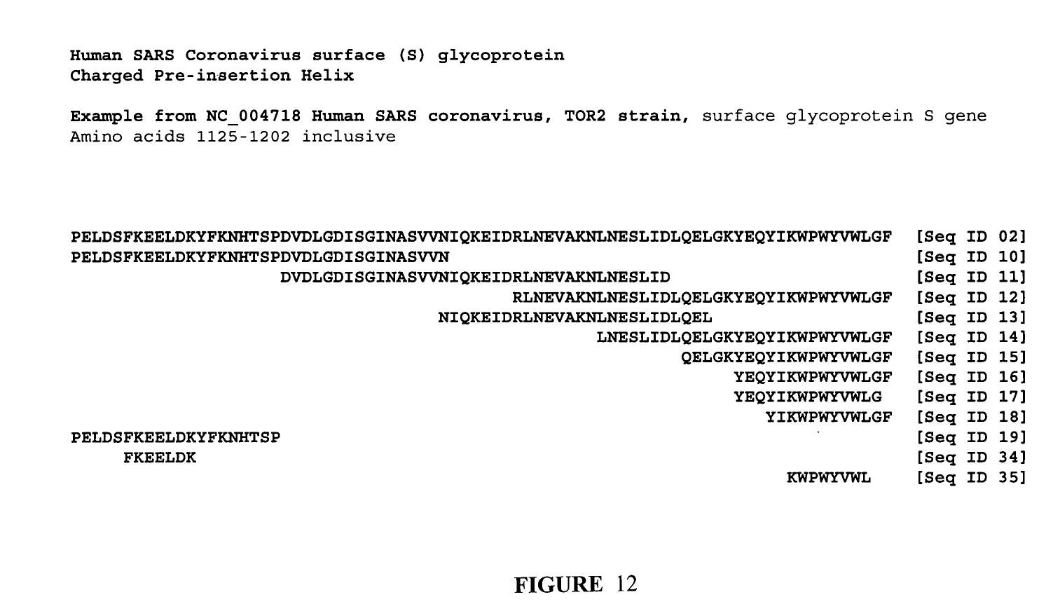

This region overlies two separate regions that meet the definition of a CPI helix, bridged by a region of lower charge density which is predicted to have a lower helicity. As in the case of human MPV, it is impracticable to synthesize or purify a peptide of this length. Therefore, 12 peptides [SEQ ID NOS: 10-19, 34 and 35] derived from this overall sequence (as shown in FIG. 12) are presented. These embodiments are to be used singly or in combination to be maximally inhibitory effective.

The following embodiment comprises a 36 amino acid peptide derived from the carboxy-terminal region of the amino acid sequence of the CPI helix which overlaps the abnormally high concentration of aromatic amino acids such as tyrosine (Y) and tryptophane (W), which have been shown to be especially active in viral fusion proteins to induce membrane fusion:

| | |
|---|---|
| RLNEVAKNLNESLIDLQEL | [SEQ ID NO: 12] |
| GKYEQYIKWPWYVWLGF. | |

Fragments of this peptide are predicted to have inhibitory effective activity, such that a sequence of as few as 10 amino acids, i.e.:

| | |
|---|---|
| YIKWPWYVWL, | [SEQ ID NO: 18] |

is predicted to yield sufficient inhibition to be effective and, at the same time, enhance ease of preparation and purification. However, the minimum effectively inhibitory peptide in the case of human CoV is either the conserved heptapeptide:

| | |
|---|---|
| FKEELDK | [SEQ ID NO: 34] |

or the conserved octapeptide:

| | |
|---|---|
| KWPWYVWL, | [SEQ ID NO: 35] |

or a combination of the two.

Each peptide has a unique and relatively poorly predictable behavior in solution. This behavior is dependent not only on the choice of the amino acid sequence, but also on the selection of molecular adducts (which could be added to the amino-terminal end and/or the carboxy-terminal end) such as any of several known to those practiced in the art useful for rendering peptides increasingly soluble, resistant to proteases, or otherwise improving their bioavailability and appropriate configuration. Desirable properties may be imparted, or undesirable properties ameliorated, by addition of adducts at either end of the proposed amino acid sequences in a manner known to those practiced in the peptide synthetic or pharmaceutical arts for development of peptide reagents for use in humans. For example, in certain embodiments of the present invention, the peptide acetyl PEQLK [SEQ ID NO: 37] is used as one of the adducts at the beginning of the peptide sequences. This addition is designed to begin the forming of (i.e., “nucleate”) the helix structure-which, once begun, will continue. The use of this additive will produce the proper helical configuration even in shorter sequences. For example, as in the following sequence:

| | |
|---|---|
| P | |
| E | |
| Q | |
| L | |
| K--- | [SEQ ID NO: 37] |

The P starts with a kink due to its ring structure, the E and Q are of high helical propensity, the L interacts with P, and the E reacts with K—all of which contribute to helix formation (Bodansky, M., Bodansky, A., The practice of peptide synthesis (2nd edn.), Springer Verlag, Berlin (1995); Gutte, B. (ed.), Peptides: Synthesis, Structure and Application, Academic Press, San Diego (1995), each of which is hereby incorporated by reference herein in its entirety).

The peptides of the present invention may be readily prepared by any of a wide range of methods known in the art, either manually or automated, while the synthetic peptide is immobilized on a solid substrate (examples can be seen in Eckert, D. M. and Kim, P. S. “Design of potent inhibitors of HIV-1 entry from the gp41 N-peptide region.” Proc Natl Acad Sci U S A. 2001 Sep. 25;98(20): 11187-92.; Giannecchini et al., 2003, “Antiviral Activity and Conformational Features of an Octapeptide Derived from the Membrane-Proximal Ectodomain of the Feline Immunodeficiency Virus Transmembrane Ectodomain”, J. Virol. 77:3724-3733; Jemmerson “Effects of Conformation, Amino Acid Sequence, and Carrier Coupling on the Immunological Recognition of Peptide and Protein Antigens” in: Zegers et al., Immunological Recognition of Peptides in Medicine and Biology (New York, CRC, 1995), pp. 213-225, each of which is hereby incorporated by reference herein in its entirety). It is anticipated that reactive side groups of the amino acids will be blocked chemically during synthesis and unblocked when synthesis is completed using methods well known to the skilled artisan. Typically, the final peptide products will be acetylated at the amino-terminal end, and amidated at the carboxy-terminal end, to increase biological half-life. Further, a D-amino acid may be interposed or added at the termini to further reduce susceptibility of the peptide to exoprotease activity in biological fluids. Any of such known methods is suitable for the present purpose.

Alternately, certain of the peptides of the present invention, especially the longer sequences (such as SEQ ID NO: 07 and SEQ ID NO: 12) may be synthesized from a genetic construct of deoxyribonucleic acid (DNA) (either synthetic or derived by duplication from the respective viral genome) that is linked to a DNA “expression vector” suitable for production of the peptide by natural or in vitro protein synthesis in a prokaryotic or eukaryotic system. A variety of expression vectors are known to those practiced in the genetic arts, and many are under continual development for a variety of genetic production methods (Kay, B., Winter, J., and McCafferty, J. Phage Display of Peptides and Proteins: A Laboratory Manual, Academic Press; 1st edition (Jan. 15, 1996), which is incorporated by reference herein in its entirety). In addition to use of DNA constructs for synthetic purposes, a contemplated application of this invention is expression of inhibitory effective peptides as a form of “gene therapy” through the administration of DNA to a human patient in lieu of the peptide itself. One example of the use of expressible DNA constructs in lieu of proteins or peptides is in immunization by injection of DNA currently under development (see Spiegelberg H L, et al. (1997) “DNA immunization: a novel approach to allergen-specific immunotherapy”, Allergy 52:964-70, which is incorporated herein by reference in its entirety).

It is contemplated that the peptides may be used singly or in combination, either with one another or with other pharmaceuticals as may be found to be compatible or synergistic. Examples of such pharmaceuticals include, but are not limited to, immune modulators such as interferon, anti-inflammatory drugs such as corticosteroids, other classes of antiviral drugs such as nucleoside analogues, or antibiotics such as erythromycin.

The peptides of the present invention may also be covalently linked, either via disulfide bridges or other chemical linkages, to each other or to macromolecular carrier molecules of desirable specificity. For example, the peptides may be linked or adsorbed to lipoproteins to facilitate their uptake into endosomal vesicles within cells as a form of biological targeting that may positively affect their efficacy. (See generally, Richard et al. (2003). Cell-penetrating Peptides A Reevaluation of the Mechanism of Cellular Uptake. The Journal of Biological Chemistry Vol. 278, No. 1, Issue of Jan. 3, pp. 585-590, which is hereby incorporated by reference herein in its entirety). Coronaviruses are known to enter cells either through direct fusion at the cell surface or via the process of endocytosis (Nash, T. C. and Buchmeier M. J. (1997). Entry of mouse hepatitis virus into cells by endosomal and nonendosomal pathways. Virology 233, 1-8; Tsai et al. (2003). Amino acid substitutions within the heptad repeat domain 1 of murine coronavirus spike protein restrict viral antigen spread in the central nervous system. Virology 312, 369-380, each of which is hereby incorporated by reference herein in its entirety). Recent studies presented at the Keystone Symposium on Bioterrorism and Emerging Infectious Diseases indicate that SARS CoV enters via endocytosis (Simmons et al. (2004). Keystone Symposium on Bioterrorism and Emerging Infectious Diseases. Abstract 215, p120, which is hereby incorporated by reference herein in its entirety) or perhaps by utilizing both cell surface and endocytic pathways as is the case with certain strains of MHV (Nash, T. C. and Buchmeier M. J. (1997). Entry of mouse hepatitis virus into cells by endosomal and nonendosomal pathways. Virology 233, 1-8; Tsai et al. (2003). Amino acid substitutions within the heptad repeat domain 1 of murine coronavirus spike protein restrict viral antigen spread in the central nervous system. Virology 312, 369-380, each of which is hereby incorporated by reference herein in its entirety). Therefore, modifications that enhance uptake of inhibitor peptides into endosomal vesicles may further increase effectiveness of the SARS CoV fusion inhibitory peptides. Certain peptides, such as Antennapedia and pestivirus Eᵣₙₛ (Garry, R. F. and Dash, S. (2003) Proteomics computational analyses suggest that hepatitis C virus E1 and pestivirus E2 envelope glycoproteins are truncated class II fusion proteins. Virology 307, 255-65, which is hereby incorporated by reference herein in its entirety) can enter cells by direct penetration of the plasma membrane. However, convincing evidence has been presented that peptides containing HIV-1 Tat amino acids 48-60 (GRKKRRQRRRP [SEQ ID NO: 38]) or polyarginine (7-9 arginines) enter cells primarily via the endocytic route (see Richard, J. P. et al. (2003). Cell-penetrating Peptides A Reevaluation of the Mechanism of Cellular Uptake. The Journal of Biological Chemistry Vol. 278, No. 1, Issue of Jan. 3, pp. 585-590, which is hereby incorporated by reference herein in its entirety). Similarly, the endosomal targeting peptide region of apolipoprotein E has been identified (Raussens, V. et al. Lipid-bound structure of an apolipoprotein E-derived peptide. J. Biol. Chem. 2003 Jul. 11;278(28): 25998-6006. Epub 2003 Apr. 22, which is hereby incorporated by reference herein in its entirety). These studies have caused a stronger consideration of the use of targeting peptide sequences, such as those found in HIV-1 Tat and human apolipoprotein E for endosomal targeting of peptide inhibitors of SARS CoV infection (see ibid.).

To determine if these endosome targeting sequences improve the efficiency of fusion inhibition of the peptides of the present invention, lead peptides will be synthesized with Tat48-60, (Arg)9 or an apolipoprotein E-derived endosomal targeting peptide (ELRVRLASHLRKLRKRLLRDADD [SEQ ID NO: 39]) at the amino or carboxyl terminus. Distribution of the modified and unmodified peptides after conjugation to Alexa Fluor 488 (spectral characteristics similar to fluorescein—excitation at 495 nm and emission at 519 nm—but produces conjugates that are brighter, more photostable, and insensitive to pH from 4 to 10) may be assessed by confocal microscopy using appropriate cell compartment tags, such as Lysotracker Red (Molecular Probes) (“Probes for Following Receptor Binding, Endocytosis and Exocytosis.” Molecular Probes Handbook., Molecular Probes, Inc., Eugene Oreg. <http://www.probes.com/handbook/sections/1601.html>; “Alexa Fluor Dyes: Simply the Best.” Molecular Probes Handbook. Molecular Probes, Inc., Eugene Oreg. <http://www.probes.com/handbook/sections/0103.html>; New Probes for Cell Tracing.” Molecular Probes Handbook. Molecular Probes, Inc., Eugene Oreg. <http://www.probes.com/lit/bioprobes25/part10.html>; Arttamangkul S, Alvarez-Maubecin V, Thomas G, Williams J T, Grandy D K. Binding and internalization of fluorescent opioid peptide conjugates in living cells. Mol Pharmacol. 2000 December; 58(6): 1570-80, each of which is hereby incorporated by reference herein in its entirety). It is predicted that the endosome targeted peptides also may inhibit CoV fusion at reduced concentrations because of increased potency.

The peptides may be suspended in any of a number of appropriate vehicles, aqueous or non-aqueous, that are pharmaceutically acceptable for human use, such as sterile solution containing other solutes (for example, sufficient saline or glucose to make the solution isotonic and compatible with human administration).

The peptides may be administered in a number of forms, to some extent depending upon the therapeutic intent. For example, one of the more useful aspects of certain embodiments of the present invention is their use prophylactically to prevent infection in those exposed or likely to be exposed to SARS-infected individuals. The peptides may be applied for either preventive or therapeutic use topically or transdermally, or by inhalation, in the form of ointments, aqueous compositions, including solutions and suspensions, creams, lotions, aerosol sprays, or dusting powders. The peptides may also be prepared and used in suppository form. The methods and applicability of such formulations is well known in the pharmaceutical art. Application of the therapeutic preparations may be to any area of the body through which the virus may be found to transmit the infection on any internal or external surface of the body, as appropriate.

The peptides may be prepared for oral or parenteral administration. In oral administration, where practicable, capsules or tablets may be prepared with stabilizers, carriers, preservatives or flavors, as is common in pharmaceutical practice. For parenteral administration, i.e., intravenous, intramuscular, subcutaneous or intraperitoneal, the peptides are administered with a pharmaceutically acceptable carrier such as a sterile solution containing other solutes or drugs.

The required dosage varies with the mode of administration. Based on our preliminary data, it appears that inhibitory effective peptides must achieve a localized concentration of 10-20 nanomolar at the site of infection. In practice, this requires administration of concentrations of peptide in micromolar quantities. Modification of the dosage range may also be dependent on whether the intent is prevention of infection or treatment of an already established infection. Such embodiments are achievable by practice of those skilled in medical arts of prevention and treatment of infectious disease. For example, clinical scientists may determine the concentration of a drug which is attained in a particular bodily fluid, such as serum, when a certain quantity of drug is administered in a certain manner and thereby adjust the dosage to attain a concentration which has been shown to be inhibitory effective in vitro.

As is known in the art, variations of the designated peptide drugs may be obtained which have superior pharmacological properties, or greater ability to inhibit evolving strains of each virus, by substituting one or more amino acids within the peptide sequence with closely related amino acids. For example, substitutions may be made within the following series of amino acids, grouped by their biochemical character:

Short side chain—Glycine (G) or Proline (P) or Alanine (A)

Hydroxylated side chain—Serine (S) or Threonine (T) or Tyrosine (Y)

Aliphatic side chain—Alanine (A) or Valine (V) or Leucine (L) or Isoleucine (1) or Methionine (M) or Cysteine (C)

Sulphur-containing side chain—Cysteine (C) or Methionine (M)

Aromatic side chain—Phenylalanine (F) or Tyrosine (Y) or Tryptophane (W)

Neutral side chain—Glutamine (Q) or Asparagine (N) or Histidine (H)

Acidic side chain—Glutamate (E) or Aspartate (D)

Basic side chain—Lysine (K) or Arginine (R)

Certain amino acids are in multiple series because they share properties with two groups of amino acids, for example, alanine is a short side chain amino acid, but also in the aliphatic series of hydrophobic side chains. The substitutions listed above are merely examples. It will be readily apparent to those skilled in the art that other substitutions are known which could be used to alter the properties of a peptide.

As an example, the amino acid sequence RIQDAIK [SEQ ID NO: 40] found in MHV is equivalent in character to the sequence RLNEVAK [SEQ ID NO: 41] in the SARS CoV, with which it may be aligned within the charged pre-insertion helix of the S2 fusion glycoprotein.

In the case of the shortest peptides of constant sequence, the shape of these peptides is critical for their activity. Such a shape can be mimicked by small organic compounds with covalent bonds that can reproduce the three dimensional shape of the natural peptide. The classic case of such a compound is penicillin, which mimics the structure of D-alanyl-D-alanine, and thus inhibits the use of that dipeptide in crosslinking bacterial cell walls as its mode of antibacterial action. While not a peptide at all, or manufactured from peptides, such compounds function as antimicrobials by mimicking the structure of peptides. Such compounds, known as peptidomimetics, may be constructed by several methods well known to those practiced in the pharmaceutical art (Hoesl C. E., Nefzi A., Ostresh J. M., Yu Y., and Houghten, R. A. Mixture-based combinatorial libraries: from peptides and peptidomimetics to small molecule acyclic and heterocyclic compounds. Methods Enzymol. 2003;369:496-517, which is hereby incorporated by reference herein in its entirety). Peptidomimetics designed or found to reproduce the structure of peptides described herein are intended to be within the scope of this invention.

The sequence and shape of the peptides defined herein can also be used to design mirror images of the peptide that would reproduce the structure of any natural ligand of the peptide. Such mirror image compounds would include peptides that complement the shape with high affinity, or antibodies directed against the peptide sequence and thus reactive with it. An example of a mirror image peptide would be regions within the antiparallel heptad repeat helix (or N-helix) of the SARS CoV, for example: ENQKQIANQFNKAISQIQESL [SEQ ID NO: 42] or KVQDVVNQNAQALNTLVKQL [SEQ ID NO: 43]. These helical sequences are similar in character to the charged pre-insertion helix, such that they would be expected to react and bind with the peptide sequences defined in the invention. Such peptides are intended to be within the scope of this invention.

An example of an antibody defined by an amino acid sequence would be an antibody designed or selected to interact with the highly conserved ELDKY [SEQ ID NO: 30] motif in the coronavirus CPI helix. Such an antibody specificity is known, the human monoclonal antibody 2F5 originally generated in the immune response to human immunodeficiency virus, type 1, which contains a highly similar ELDKW [SEQ ID NO: 31] motif in its CPI helix region. Use of such an antibody, that reacts with CPI helix peptides and is used in lieu of such peptides, is also intended to be within the scope of this invention.

It is a contemplated application of the present invention that peptides be tested initially by testing comparable peptides of animal viruses or less virulent strains of human viruses, and that permanent lines of animal and human cells in culture be used both as host cells for experimental infections, as well as for toxicity testing. Such testing systems prevent the endangerment of personnel by exposure to virulent human pathogenic viruses such as the SARS CoV. Combinations of such testing systems include the OC43 strain of the human CoV in infection of the Vero E2 permanent cell line of African green monkey kidney cells (American Type Culture Collection, Manassas, Va.). Peptides from the comparable CPI helix of OC43 are derived from the region:

| | |
|---|---|
| PNLPDFKEELDQWFKNQTSVAPDLSLDYINVTLD | [SEQ ID NO: 20] |
| LQVEMNRLQEAIKVLNQSYINLKDIGTYEYYVKW | |
| PWYVW. | |

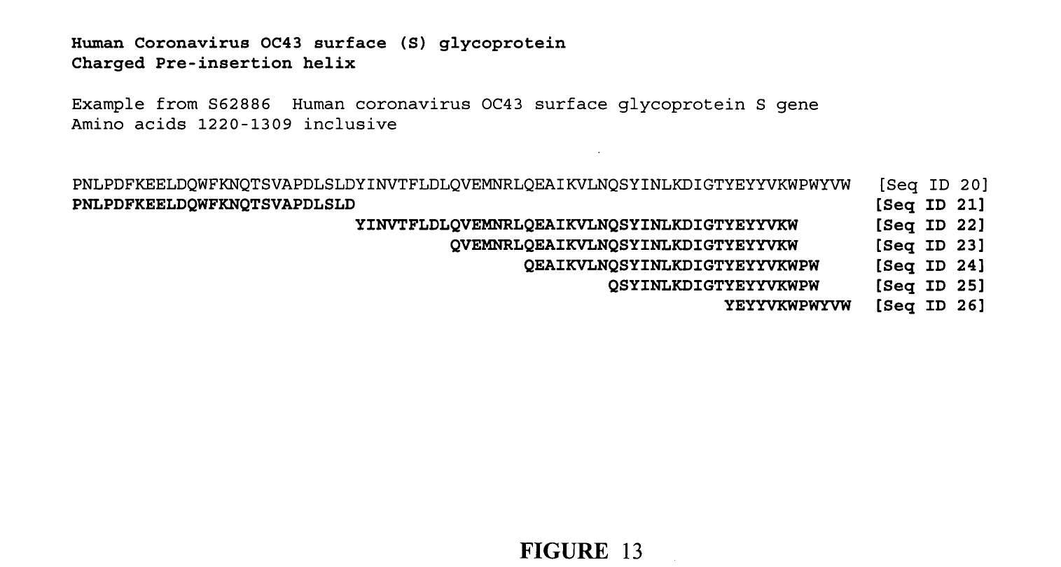

Peptide analogues of OC43 corresponding to peptide analogues of human SARS CoV include SEQ ID NOS: 21-26, the relationship of which to SEQ ID NO: 20 is shown in FIG. 13.

Briefly, Vero E2 cells are treated with an inhibitory effective concentration of peptide to equilibrate the culture system with solution containing peptide. A solution containing OC43 human coronavirus is then added, in the continued presence of the peptide solution. Comparable mock-treated controls are allowed to be infected normally as a positive control, and uninfected controls are treated with peptide continuously in the absence of virus, as a control for toxicity. Other control cultures are continuously treated with solution containing neither peptide nor virus, as a negative control. The effects of infection are measured both by observation of cellular cytopathology as a result of virus multiplication, as well as by noting the yield of progeny virus by any of a variety of molecular and virological means well known to virologists practiced in the art. Such studies generally follow the prototype of peptide inhibition studies established in studies of influenza and measles viruses (Richardson, C. D. et al. 1980. Specific inhibition of Paramyxovirus and myxovirus replication by oligopeptides and amino acid sequences similar to those at the N-termini of the Fl or HA2 viral polypeptides. Virology 105, 205-222.; Hsu, M. C. et al. 1981. Activation of the Sendai virus Fusion protein (F) involves a conformational change with exposure of a new amino terminus. Virology 104, 294-302.; Richardson, C. D. and Choppin, P. W. 1983. Oligopeptides that specifically Inhibit membrane fusion by paramyxoviruses: studies on the site of action. Virology 131, 518-532, each of which is hereby incorporated by reference herein in its entirety).

Given the reduced cytopathology inherent in the OC43 virus, and the general observation of only limited human disease due to OC43, testing of peptides for human use may include the use of experimental infections of humans with OC43, and its prevention or treatment by inhibitory effective dosages of peptides targeted to the OC43 CPI helix sequence of amino acids. Such testing may yield critical information preparatory to clinical trials utilizing peptide drugs targeted against the more virulent and cytopathogenic SARS CoV. Insofar as viruses similar to or identical with OC43 are responsible for human illness such as the common cold, the peptides of this invention may be useful for prevention or treatment of such mild respiratory infections, either alone or in combination with other antiviral drugs or other medications. It is contemplated that the same variations in formulation or delivery may be utilized as described above for the formulations involving peptides targeted against human metapneumovirus or human SARS coronavirus.

Prior to, in lieu of, or to supplement testing with OC43 coronavirus, animal testing is typically performed in vitro, using an appropriate combination of animal virus and animal cell line, or in vivo, using an appropriate animal host. In the case of coronaviruses, a widely established and useful system is that of the MHV in an established permanent line of mouse cells, L2 (American Type Culture Collection, Manassas, Va.), or in experimental infection of mice. Particularly useful is a cytopathogenic strain of MHV, A59, which has been used to study coronavirus induced cell fusion. The peptide region of the S2 glycoprotein to MHV A59 that is similar to the comparable portion from the human SARS CoV is the following peptide, which was taken from the CPI helix of MHV A59 S2 glycoprotein:

| | |
|---|---|
| QDAIKKLNESYINLKEVGTYEMYVKWPWYVW. | [SEQ ID NO: 27] |

This model peptide is useful as a “proof of concept” peptide, due to its similarity to the comparable region of the human SARS CoV S2 glycoprotein, and due to the fact that MHV A59 is comparably cytopathic in mouse L2 cells, as the SARS CoV is in human cells. This peptide provides a close parallel system that is innocuous to humans but may be utilized to test the full spectrum of toxicity, bioavailability, stability and optimal dosage of the present invention, without endangerment of humans or restriction of studies to specialized biological safety environments.

To test the unique properties of each inhibitory effective peptide, additional controls to be tested include peptides of equal length and composition to the peptides of this invention, but with the order of amino acids scrambled in random order. The specificity of each peptide is also contemplated to be tested by testing peptides derived from one virus sequence on other viruses with different sequences. Each sequence is unique to each virus, with considerable variation even among closely related viruses in the same family. Optimal peptides for each virus system vary in their position within the CPI helix sequence motif relative to the membrane-spanning domain. Nevertheless, specificity will be demonstrated by testing irrelevant peptide compositions and sequences.

### Examples

A. Inhibitory Peptides

Preliminary Studies indicate that peptide inhibitors can be developed for members of the Coronaviridae family of viruses. We have tested synthetic peptides for their ability to inhibit plaque formation by MHV. We have observed that certain peptides inhibit plaque formation by MHV, and we have confirmed these results for selected inhibitory and non-inhibitory peptides. We found that a peptide corresponding to the MHV C-helix having the following sequence:

RIQDAIKKLNESYINLKEVGTYEMYVKWPWYVWLLI (SEQ ID NO: 52)

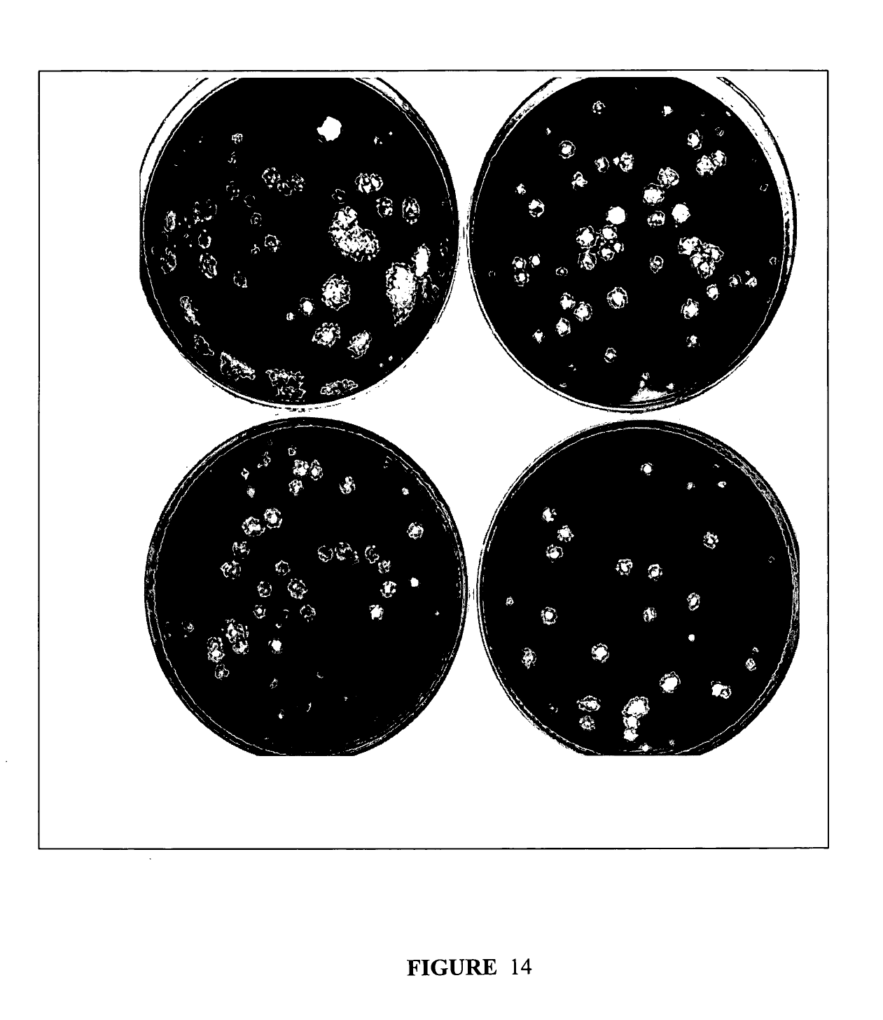

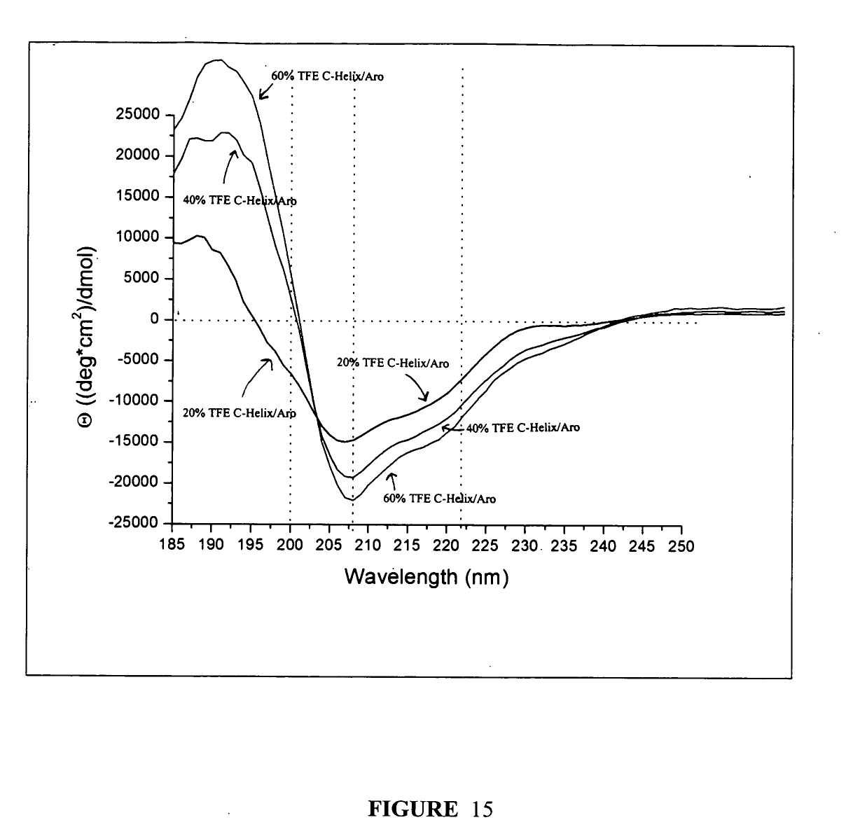

reduced plaque formation by about 40% at a nominal concentration of about 25 μM (see FIG. 14). There was also a significant reduction (about 50%) in the average diameter of the plaques. These results suggest that this peptide inhibits both entry and spread of MHV. Similar results with this inhibitory peptide were obtained in two additional independent experiments, with significant plaque inhibition observed at concentrations of as low as 1 μM. These results are unlikely to be explained by non-specific cytotoxic effects of the peptide. Killing the cells would inhibit fusion, but the cells in these studies have normal morphology, indicating they are unlikely to be damaged to an extent that would inhibit them through any toxic effect. Except for the plaques, cells in the monolayers were intact and viable, and the low number of plaques that did grow were similar in size to control plaques. Comparable results, with inhibitory activities in the μM range have been reported with a C-helix peptide (Bosch, B. J. et al. (2003). The coronavirus spike protein is a class I virus fusion protein: structural and functional characterization of the fusion core complex. J Virol 77, 8801-8811, which is hereby incorporated by reference herein in its entirety). Preliminary experiments also indicate that these peptides form helical structures in aqueous solution which are responsible for their biological function as inhibitors. FIG. 15 shows the results of Circular dichroism (CD) spectroscopy used to delineate the structural properties of a peptide corresponding to a region of the S2 protein of MHV encompassing a portion of the C-helix and the aromatic domain (SEQ ID NO: 52). Collectively, these results suggest that our approaches can identify synthetic peptides that inhibit fusion/infectivity by members of the Coronaviridae family (see also Tripet, B. et al. (2004). Structural Characterization of the SARS-Coronavirus Spike S Fusion Protein Core. JBC Papers in Press. Manuscript M400759200; Liu, S. et al. (2004). Interaction between heptad repeat 1 and 2 regions in spike protein of SARS-associated coronavirus: implications for virus fusogenic mechanism and identification of fusion inhibitors. The Lancet Vol. 363, pp. 938-940, each of which is hereby incorporated by reference herein in its entirety).

1. Procedures

a. CD Spectroscopy

As noted, to examine the potential for the formation of secondary structures upon interaction with lipid membranes, peptides were examined by CD spectroscopy. Circular dichroism (CD) spectra were recorded on a Jasco J-810 spectrapolarimeter (Jasco Inc., Easton, Md.), using a 1 mm path length, 1 nm bandwith, 16 second response time and a scan speed of 10 nm/min. All CD runs were performed at room temperature with peptide dissolved in 10M potassium phosphate buffer at pH 7.0. LUV were added at a lipid concentration of 1 mM from a stock in 10 mM potassium phosphate buffer pH 7.0. Three successive scans between 190-250 nm were collected and the CD data (see FIG. 15) are expressed as the mean residue ellipticity, derived from the formula θ=(deg*cm2)/dmol (see Wimley, W. C., and White, S. H. (2000). Designing transmembrane alpha-helices that insert spontaneously. Biochemistry 39, 4432-42, which is hereby incorporated by reference herein in its entirety).

2. Viral Plaque Assays

L2 cells were maintained as monolayers in complete Dulbecco's modified Eagle's medium (DMEM) containing 0.15% HCO3-supplemented with 10% fetal bovine serum (FBS), penicillin G (100 U/ml), streptomycin (100 mg/ml), and 2 mM L-glutamine at 37° C. in a 5% CO₂ incubator. MHV strain A59 (ATCC, VR764) was propagated on L2 cells as described in Compton S. R., Winograd D. F., Gaertner D. J. Optimization of in vitro growth conditions for enterotropic murine coronavirus strains. J Virol Methods. 1995 April; 52(3): 301-7, which is hereby incorporated by reference herein in its entirety. For plaque assays, L2 cells were seeded at a density of 1×10⁶ cells in each well of a 6-well plate. Approximately 100-plaque forming units (p.f.u.) of MHV were pre-incubated with or without 100 μg/ml of inhibitory peptide (SEQ ID NO: 52) in serum-free DMEM for 1 h. L2 cells were then infected with peptide-treated inoculum or vehicle control inoculum. After 1 hour adsorption, the inoculum was removed, cells were washed twice with 1× phosphate buffered saline, and the cells were overlaid with 10% FBS/DMEM containing 0.5% SeaPlaque Agarose (Cambrex Bio Science Rockland, Inc., Rockland, Me.). Monolayers were fixed with 3.7% formalin and stained with 1× crystal violet 2 days post-infection, and plaque numbers were determined by light microscopy (Haff, R. F. (1962) Plaque formation by a mouse hepatitis virus. Virology 18, 507-508, which is hereby incorporated by reference herein in its entirety.)

Results of the viral plaque assay using the peptide having the sequence of SEQ ID NO 52 are illustrated in FIG. 14. The upper wells are controls exposed to vehicle and the lower wells exposed to the peptide at a nominal concentration of 25 μm. Plaques were visualized after 3 days by staining cells with crystal violet. The results show that the peptide reduced plaque formation by about 40%. There was also significant reduction (about 50%) in the average diameter of the plaques. These results suggest that this peptide inhibits both entry and spread of MHV.

B. Biophysical Experiments

1. Interfacial Hydrophobicity Analysis

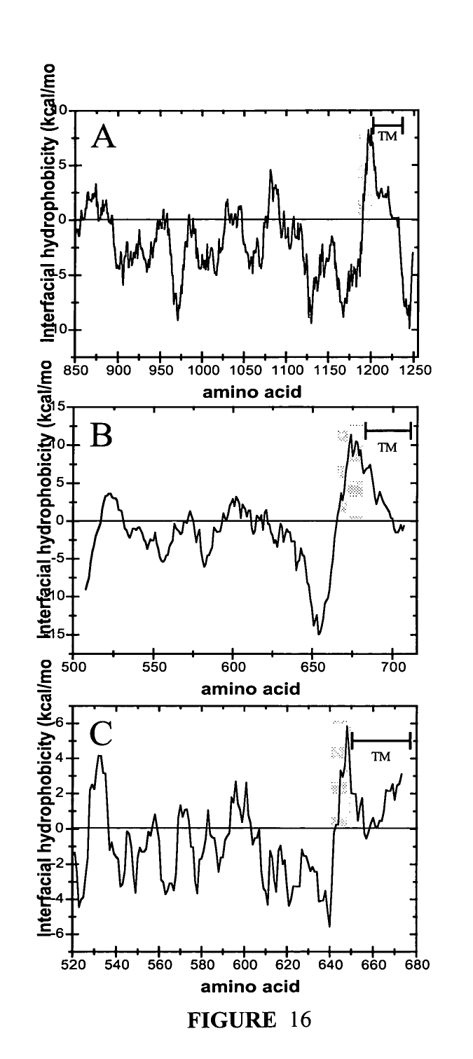

The Wimley and White hydrophobicity-at-interface scale was used to identify regions of the CoV fusion glycoprotein with high propensity to partition into lipid membranes. This scale is based on the free energies of transfer DG (kcal/mol) of amino acid sequences from water into bilayer interfaces and n-octanol, taking into consideration the contribution from the peptide bond (Wimley, W. C., Selsted, M. E., and White, S. H. (1994). Interactions between human defensins and lipid bilayers; evidence for formation of multimeric pores. Protein Sci 3, 1362-73; Wimley, W. C. and White, S. H. (2000a). Designing transmembrane alpha-helices that insert spontaneously. Biochemistry 39, 4432-42; Wimley, W. C. and White, S. H. (2000b). Determining the membrane topology of peptides by fluorescence quenching. Biochemistry 39, 161-70, each of which is hereby incorporated by reference herein in its entirety). Due to the salient similarities between the CoV fusion glycoprotein and the class I fusion glycoproteins of other RNA viruses, we compared the interfacial hydrophobicity plots of SARS CoV fusion glycoprotein to the fusion glycoproteins of HIV-1 gp41 and Ebola virus. When average interfacial hydrophobicity was plotted for the fusion proteins of these three viruses, similar regions with high propensity for membrane partitioning were detected. At the N-terminal region of all three fusion glycoproteins, a region of high interfacial hydrophobicity was detected. For HIV-1 and Ebola virus, this region corresponds to the viral fusion peptide (see FIG. 16B and FIG. 16C). Although no putative fusion peptide has been determined for the SARS CoV fusion glycoprotein, a stretch of 19 hydrophobic amino acids (WTFGAGAALQIPFAMQMAY [SEQ ID NO 51]) with an average interfacial hydrophobicity score of 2.42 kcal/mol was detected as the N-terminal region of the fusion glycoprotein. The location of this region is almost coincident with that of the HIV-1 and Ebola virus fusion peptides, and should therefore be considered as a possible fusion protein of the SARS-CoV S protein.

A second region of high interfacial hydrophobicity was detected at the C-terminal end of the fusion glycoproteins, correlating to the putative transmembrane domain of the SARS CoV fusion glycoprotein (residues 1190-1225 of FIG. 16A), and the experimentally determined membrane spanning anchors of HIV-1 gp41 and Ebola virus GP2 (residues 665-700 of FIG. 16B and residues 644-672 of FIG. 16C, respectively). Nieva and colleagues have shown that for HIV-1 and Ebola virus, this large region of high interfacial hydrophobicity is segmented into two-independent domains: one aromatic amino acid rich domain lying within the C-terminal end of the fusion protein and a second domain comprising the membrane-spanning anchor of the fusion protein (Nieva, J. L. et al. (1994). Interaction of the HIV-1 fusion peptide with phospholipid vesicles: different structural requirements for fusion and leakage. Biochemistry 33, 3201-9, which is hereby incorporated by reference herein in its entirety). The hydrophobic region at the C-terminal end of the SARS CoV fusion glycoprotein shows a remarkable similarity to that of the HIV-1 gp41 and Ebola virus GP2 in that a region of aromatic amino acids is also present and proximal to the transmembrane domain. Due to the high interfacial propensity of the aromatic region alone (3.58 kcal/mol), it is unlikely that this region is part of the transmembrane anchor as previously predicted by Rota et al. (Rota P. A. et al. Characterization of a novel coronavirus associated with severe acute respiratory syndrome. Science. 2003 May 30, which is hereby incorporated by reference herein in its entirety). Rather, like the aromatic domains of HIV-1 and Ebola virus, this region is most likely an independent domain proximal to the transmembrane anchor of the fusion glycoprotein.

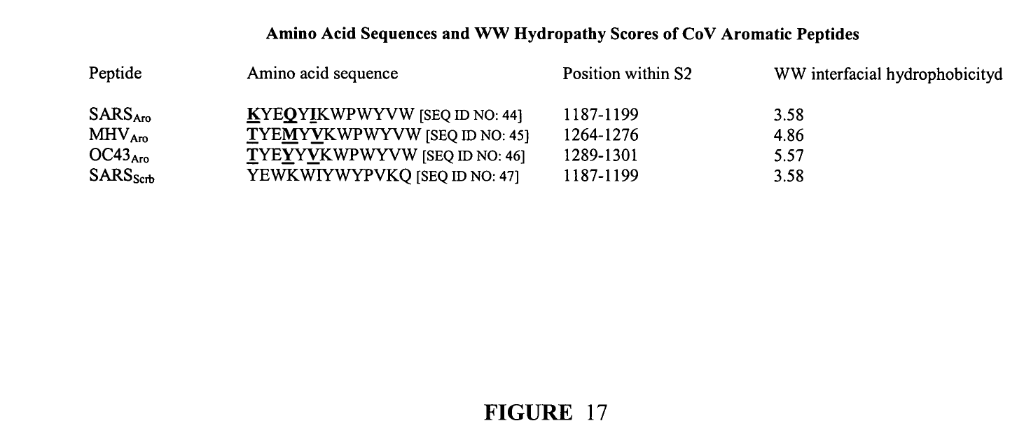

Sequence analysis of the fusion glycoprotein of MHV and the human CoV OC43 showed coinciding interfacial hydrophobicity plots to that of the SARS CoV fusion glycoprotein. In addition, the presence of highly-conserved aromatic domains, differing in only 3 amino acids to the SARS aromatic domain, were identified (see FIG. 17). Interfacial hydrophobicity scores of 3.58, 4.86 and 5.57 kcal/mol were predicted for the aromatic domains of SARS CoV, MHV, and OC43, respectively. Based on the these analyses, peptides of 13 amino acids in length were synthesized and used throughout this study to determine the functional importance of this region within the CoV fusion glycoprotein.

2. Peptide Synthesis

The following peptides were synthesized by solid-phase methodology using a semi-automated peptide synthesizer and conventional N-alpha-9-fluorenylmethyloxycarbonyl (Fmoc) chemistry by Genemed Synthesis, Inc. (San Francisco, Calif.):

| | |
|---|---|
| (SARS_{Aro}) | |
| KYEQYIKWPWYVW | [SEQ ID NO: 44] |
| (MHV_{Aro}) | |
| TYEMYVKWPWYVW | [SEQ ID NO: 45] |
| (OC43_{Aro}) | |
| TYEYYVKWPWYVW | [SEQ ID NO: 46] |

SARS-CoV scrambled peptide (SARS_{Scr}) YEWKWIYWYPVKQ [SEQ ID NO: 47] The SARS aromatic (SARS_{Aro}), MHV aromatic (MHV_{Aro}) and OC43 aromatic (OC43_{Aro}) (collectively referred to sometimes as the “CoV aromatic peptides”) were synthesized based on their amino acid sequence determined from GenBank accession no. AY278741 (SARS-CoV strain Urbani), AY497331 (MHV strain A59), and NP_{—}937950 (Human CoV OC43). The SARS_{Aro} sequence was arbitrarily scrambled to generate the peptide SARS_{Scr}. Hydropathy scores were determined according to methods known in the art using the Wimley and White (WW) interfacial hydrophobicity scale using a window of 13 residues (see FIG. 17). Peptides were purified by reversed-phase high performance liquid chromatography, and their purity confirmed by amino acid analysis and electrospray mass spectrometry. Peptide stock solutions were prepared in DMSO (spectroscopy grade), and concentrations determined spectroscopically (SmartSpec™ 3000, BiORad, Hercules, Calif.).

3. CoV Aromatic Domains Interact with Lipid Membranes

We first assessed the ability of the CoV aromatic peptides to interact with membranes of large unilamellar vesicles (LUV) composed of different lipid compositions. LUV composed of 1-palmitoyl-2-oleyl-sn-glycero-3-phosphocholine (POPC) with phosphatidylinositol (PI), 1-palmitoyl-2-oleyl-sn-glycero-3-[phosphor-rac-(1-glycerol)] (POPG) and/or cholesterol (CHOL) were used as targets in partitioning experiments with the CoV aromatic peptides. The degree to which a peptide partitions into a vesicle can be determined fluorometrically by observing the change in tryptophan fluorescence (F) as a function of increasing lipid titration. The fluorescence of tryptophan increases in the presence of a low-polarity environment, such as the lipid membrane interface. Based on the average interfacial hydrophobicity scores of each CoV aromatic peptide alone, we predicted that all of the CoV aromatic peptides would partition into the membranes of the target vesicles.

a. LUV Preparation

Large unilamellar vesicles (LUV) consisting of POPC with POPG, PI (Avanti Polar Lipids, Birmigham, Ala.) and/or cholesterol (Sigma, St. Louis, Mich.) were prepared according to the extrusion method of Mayer, et al (Mayer L. D., Hope M. J., Cullis P. R. Vesicles of variable sizes produced by a rapid extrusion procedure. Biochim Biophys Acta. 1986 Jun. 13;858(1):161-8, which is hereby incorporated by reference herein in its entirety). Briefly, lipids were dried from chloroform solution with nitrogen gas stream and high vacuum overnight. Lipid vesicles used in peptide binding assays and CD experiments were resuspended in 10 mM potassium phosphate buffer to bring the concentration to 100 mM total lipid. Samples were subjected to repeated freeze and thaw for 15 cycles followed by extrusion through 0.1 m polycarbonate membranes in a Lipex Biomembranes extruder (Lipex Biomembranes, Vancouver BC). To prepare Tb3+LUV, lipids were resuspended to 100 mM concentration in 50 mM Tb3+, 100 mM sodium citrate, and 10 mM TES pH 7.2. Gel filtration on Sephadex G-200 was used to remove unencapsulated terbium in a buffer of 10 mM TES and 325 mM NaCl (56). LUV were eluted from a Sepadex G-200 gel column using 10 mM potassium phosphate pH 7.0. Final lipid concentrations were determined by phosphate analysis.

b. Interaction Assay

Partitioning of peptides into lipid bilayer was monitored by the fluorescence enhancement of tryptophan (White, S. H., Wimley, W. C., Ladokhin, A. S., and Hristova, K. (1998) Protein folding in membranes: determining energetics of peptide-bilayer interactions. Methods Enzymol 295, 62-87, which is hereby incorporated by reference herein in its entirety). Fluorescence was recorded at excitation and emission wavelengths of 280 nm and 340 nm, respectively, and 8 nm bandwidths using an SML Aminco 8100 spectrofluorometer (Rochester, N.Y.). Quartz cuvettes were used with excitation and emission path lengths of 4 mm and 10 mm. Measurements were carried out in 10 mM potassium phosphate pH 7.0. Peptides were added from DMSO stock solutions to 250 μl of buffer and mixed by inversion. LUV at a final lipid concentration of 1 mM were titrated into solution and mixed by inversion. Intensity values (I) were adjusted for lipid scattering and normalized to peptide in buffer (Io). Partitioning coefficients were obtained by fitting the formula:

I/Io=1+(((Kx*[L])/([W]+(Kx*[L])))*((Imax/Io)−1)

to the normalized data using 55.3M for water ([W]) and where Imax is equal to peptide signal at 1 mM lipid (Wimley, W. C., and White, S. H. (2000). Designing transmembrane alpha-helices that insert spontaneously. Biochemistry 39, 4432-42, which is hereby incorporated by reference herein in its entirety).

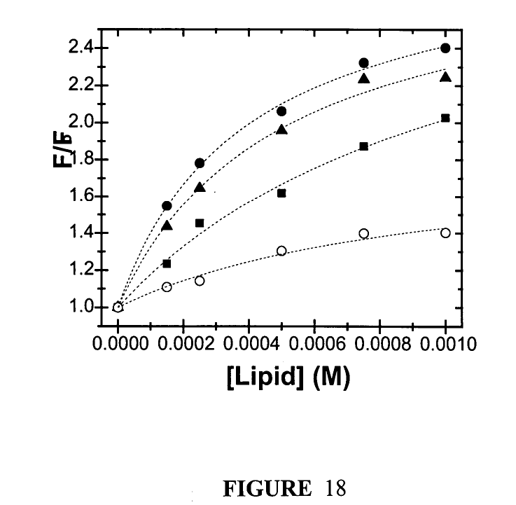

FIG. 18 shows the normalized tryptophan fluorescence (F/Fo) for the SARS_{Aro} peptide as a function of increasing lipid concentration of different LUV (mM). SARS_{Aro} fluorescence increased as a direct function of increasing lipid concentrations of LUV composed of POPC. A more significant increase in tryptophan fluorescence was observed when LUV composed of POPC and either PI or POPG were titrated with the peptide, suggesting an intrinsic role for anionic lipids as a part of the membrane composition. This effect, however, was reduced when cholesterol was included as part of the membrane composition of POPC:PI LUV, perhaps due to its ability to rigidify lipid membranes. As predicted, all four CoV peptides examined partition into lipid membranes. The degree of partitioning for all four peptides was similar, and the presence of anionic lipids in the membrane composition enhanced peptide partitioning, as seen in FIG. 18. The addition of CHOL, however, inhibited peptide partitioning, most notably with POPC:CHOL LUV and to a lesser extent with POPC:PI:CHOL and POPC:POPG:CHOL LUV.

4. Tb3+/DPA Microwell Assay

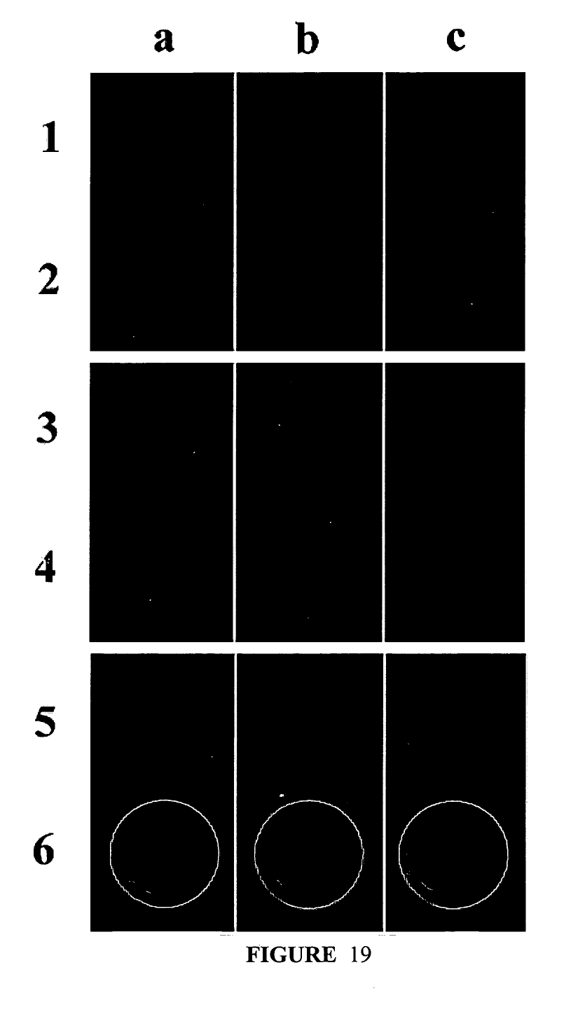

To test the potential of the CoV aromatic peptides to perturb membrane integrity, a high-throughput leakage assay was used. The Tb3+/DPA microwell assay is a sensitive visual screening assay known in the art to rapidly identify peptides capable of permeabilizing lipid membranes (see Rausch, J. M., and Wimley, W. C. (2001) Anal Biochem 293, 258-263, which is hereby incorporated by reference herein in its entirety). The detectability is based on the strong fluorescence emission of the lanthanide metal Tb3+ when it interacts with the aromatic chelator DPA. In the experimental assay, CoV aromatic peptides were incubated at peptide:lipid molar ratios of 1:100 and 1:50 with 500 mM lipid. After 2 h incubation at room temperature, the extent of Tb3+ leakage from lipid vesicles was visually determined by the detection of a bright green fluorescence upon irradiation with UV light. An example plate is shown in FIG. 19 in which the SARS_{Aro} (rows 1 and 2) and SARS_{Scr} (rows 3 and 4) peptides were tested for their potential to permeabilize LUV composed of POPC, POPC:PI (9:1) or POPC:POPG (9:1). The SARS_{Aro} peptide at peptide:lipid ratios of 1:100 and 1:50 permeabilized all three LUV tested, with the greatest degree of fluorescence detected in wells with POPC or POPC:PI (9:1) LUV. In contrast, the SARS_{Scr} peptide did not induce leakage of any of the three LUV tested, as detectable by this assay. The extent of leakage induced by SARS_{Aro} was less than the observed leakage in the detergent solubilized wells (row 6). Comparable results were achieved with the MHV_{Aro} and OC43_{Aro} peptides at peptide:lipid ratios of 1:100 and 1:50, with OC43_{Aro} exhibiting the slightly lower levels of leakage (data not shown).

5. ANTS-DPX Leakage Assay

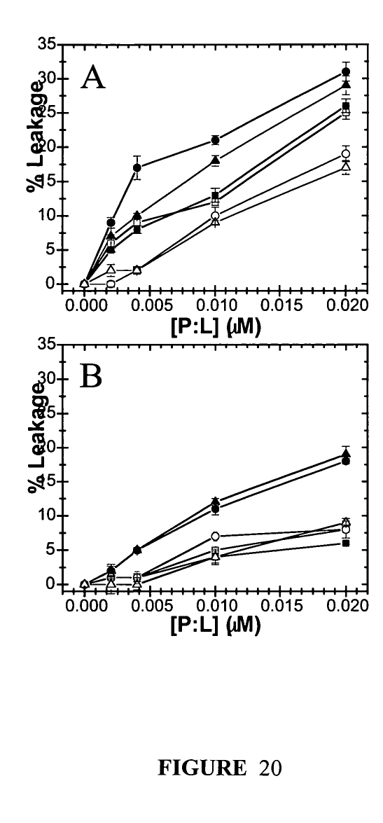

We employed the use of the ANTS/DPX leakage assay as a second means of determining the membrane permeabilization capacity of the CoV aromatic peptides. The ability of the SARS_{Aro} and SARS_{Scr} peptides to release the fluorescent probe ANTS encapsulated within LUV was examined at peptide to lipid ratios of 1:500, 1:250, 1:100 and 1:50. As with the Tb3+/DPA microwell assay, the SARS_{Aro} peptide induced leakage of ANTS from LUV to a greater degree than its scrambled counterpart, SARS_{Scr} (see FIG. 20) On average, the percent leakage detected at all peptide:lipid ratios was approximately 2 to 3 times greater for the SARS_{Aro} peptide as compared to the SARS_{Scr} peptide (FIG. 20). The degree of leakage induced by SARS_{Aro} varied based on the lipid composition of the LUV tested. The percent leakage detected from LUV composed of either POPC:PI or POPC:POPG was 25% and 22%, respectively, as compared to 15% leakage observed in POPC LUV at peptide:lipid ratios of 1:100 (FIG. 20).

6. CD Spectroscopy

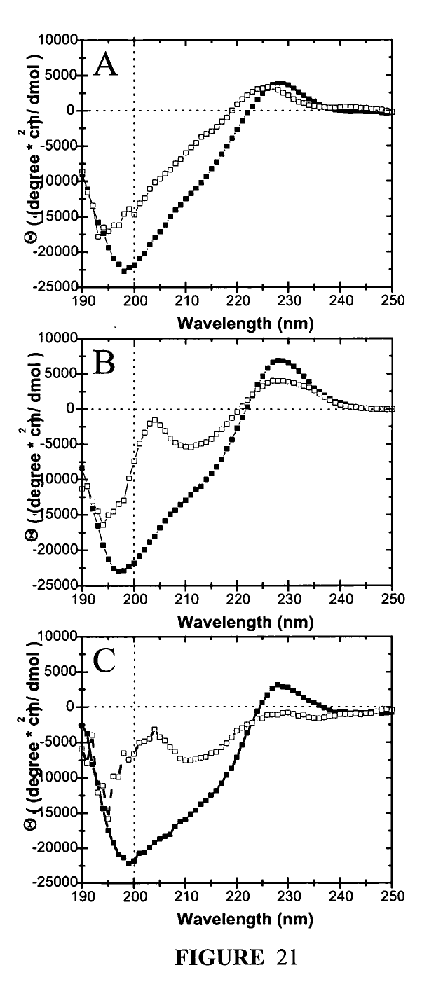

To examine the potential for the formation of secondary structures upon interaction with lipid membranes, the CoV aromatic peptides were examined by CD spectroscopy. Circular dichroism (CD) spectra were recorded on a Jasco J-810 spectrapolarimeter (Jasco Inc., Easton, Md.), using a 1 mm path length, 1 nm bandwith, 16 second response time and a scan speed of 10 nm/min. All CD runs were performed at room temperature with peptide dissolved in 10 mM potassium phosphate buffer at pH 7.0. LUV were added at a lipid concentration of 1 mM from a stock in 10 mM potassium phosphate buffer pH 7.0. Three successive scans between 190-250 run were collected and the CD data (see FIG. 21) are expressed as the mean residue ellipticity, derived from the formula θ=(deg*cm2)/dmol (Wimley, W. C., and White, S. H. (2000). Designing transmembrane alpha-helices that insert spontaneously. Biochemistry 39, 4432-42, which is hereby incorporated by reference herein in its entirety).

The results of the CD spectroscopy study are shown in FIG. 21, which illustrates representative far UV CD spectra of the CoV aromatic peptides in buffer and with LUV. Analysis of the CoV peptides in 10 mM PO4 buffer pH 7.0 showed a random coil spectrum with single minima at 200 nm. No defined α-helical or β-sheet structure was apparent for any of the three CoV peptides in buffer alone. We next analyzed the potential of the CoV aromatic peptides to adopt a secondary structure in the presence of lipids. Results from our peptide partitioning and vesicle leakage assays suggested that the CoV aromatic peptides preferentially interacted with LUV composed of POPC and anionic lipids. We therefore analyzed the UV CD spectra of the CoV aromatic peptides with LUV composed of POPC:PI at lipid concentrations of 1 mM. Again, no defined secondary structure was apparent for any of the three CoV peptides in the presence of lipid. For the MHV_{Aro} and OC43_{Aro} peptides, however, there was a distinct change in the observed CD spectra as compared to buffer alone (see FIGS. 21B and 21C). Although not indicative of a defined secondary structure due to the lack of minima at 208 nm and 222 nm for α-helical structures or 218 nm for β-sheet structures, it appears that the peptides may be assuming a more ordered structure above that of a random coil. These results are not surprising as the CoV aromatic peptides are only 13 amino acids long, a length not sufficient to cross a lipid membrane (see Rausch J. M. and Wimley W. C. (2001). A high-throughput screen for transmembrane pore-forming peptides. Analytical Biochemistry 293:258-63, which is hereby incorporated by reference herein in its entirety).

What is claimed is:

1. A peptide derived from an enveloped virus having a fusion glycoprotein amino acid residue sequence and a CPI helix amino acid residue subsequence in said fusion glycoprotein amino acid residue sequence, said peptide corresponding to a segment of said CPI helix amino acid residue subsequence.

2. A peptide according to claim 1 wherein said enveloped virus is human metapneumovirus.

3. A peptide according to claim 1 wherein said enveloped virus is human coronavirus.

4. A peptide according to claim 1 wherein said peptide is derived from a segment of said CPI helix amino acid residue subsequence that is conserved among class I viral fusion glycoproteins.

5. A peptide according to claim 1, wherein said peptide comprises an amino acid residue sequence chosen from the group consisting of SEQ ID NO: 1 through SEQ ID NO: 26, SEQ ID NO: 34 and SEQ ID NO: 35.

6. A peptide according to claim 1, wherein said peptide comprises an amino acid residue sequence comprising SEQ ID NO: 52.

7. The peptide of claim 5, wherein said peptide further comprises an adduct (x) at the amino terminus of said peptide or an adduct (x′) at the carboxy terminus of said peptide.

8. The peptide of claim 6, wherein said adduct x is selected from the group consisting of an acetyl group, a carbobenzoxy group, a 9-fluorenylmethoxy group, a D-amino acid, a hydrophobic adduct, a carrier macromolecule, a lipid, and the peptide acetyl PEQLK [SEQ ID NO: 37].

9. The peptides of claim 6 wherein the adduct x′ is selected from the group consisting of an amido group, a hydrophobic adduct, a carrier macromolecule, and a lipid.

10. A method for inhibiting infection of a human cell by human metapneumovirus comprising administering to a human host an inhibitory effective concentration of a peptide, wherein said peptide comprises a segment from a CPI helix amino acid residue subsequence of a fusion glycoprotein amino acid residue sequence of said human metapneumovirus.

11. The method according to claim 9, wherein said peptide is derived from SEQ ID NO. 01.

12. A method for inhibiting infection of a human cell by human coronavirus comprising administering to a human host an inhibitory effective concentration of a peptide, wherein said peptide comprises a segment from a CPI helix amino acid residue subsequence of a fusion glycoprotein amino acid residue sequence of said human coronavirus.

13. The method according to claim 11 wherein said peptide comprises a segment from SEQ.ID NO. 02 or SEQ ID NO. 20.

14. A method for inhibiting infection of a human cell by human metapneumovirus comprising administering to a human host an inhibitory effective concentration of a combination of peptides, wherein each of said peptides in said combination of peptides comprises a segment from a CPI helix amino acid residue subsequence of a fusion glycoprotein amino acid residue sequence of said human metapneumovirus.

15. The method according to claim 13 wherein each of said peptides in said combination of peptides comprises a segment from SEQ ID NO. 01.

16. A method for inhibiting infection of a human cell by human coronavirus comprising administering to a human host an inhibitory effective concentration of a combination of peptides wherein each of said peptides in said combination of peptides comprises a segment from a CPI helix amino acid residue subsequence of a fusion glycoprotein amino acid residue sequence of said human coronavirus.

17. The method according to claim 15 wherein each of said peptides in said combination of peptides comprises a segment from SEQ ID NO: 02 or SEQ ID NO: 20.

18. A method for inhibiting infection of a human cell by human coronavirus comprising administering to a human host an inhibitory effective concentration of a combination of peptides wherein each of said peptides in said combination of peptides comprises a segment from the RNA of said virus corresponding to a CPI helix amino acid residue subsequence of a fusion glycoprotein amino acid residue sequence of said human coronavirus.

19. A process for selecting a peptide as a candidate for inhibiting infection of a human cell by an enveloped virus having a fusion glycoprotein amino acid residue sequence and a CPI helix amino acid residue subsequence in said fusion glycoprotein amino acid residue sequence, comprising:
(1) searching the primary amino acid residue sequence of said virus for an amino acid subsequence 1 of about 20-25 amino acid residues containing more than about 60 percent of hydrophobic amino acid residues (Phenylalanine (F), Tyrosine (Y), Tryptophane (W), Alanine (A), Valine (V), Leucine (L), Isoleucine (I), Methionine (M) or Cysteine (C));
(2) searching within a range of about 100 amino acid residues from the amino end of said subsequence 1 for a subsequence 2 containing more than about 60% of: (a) charged amino acid residues (Glutamate (E) or Aspartate (D), Lysine (K) or Arginine (R)) and (b) Helix amino acids residues (glutamine (Q), glutamate (E), alanine (A), phenylalanine (F), tryptophane (W), lysine (K) or leucine (L));
(3) selecting a subsequence 3 of said subsequence 2.

20. The process of claim 18 further including the step of testing said subsequence 3 for inhibitory effectiveness.

21. The process of claim 18 wherein said wherein amino acid residues in said subsequence 3 are further substituted with alternate amino acid residues having similar biological properties, as follows:
Short side chain—Glycine (G) or Proline (P) or Alanine (A)
Hydroxylated side chain—Serine (S) or Threonine (T) or Tyrosine (Y)
Aliphatic side chain—Alanine (A) or Valine (V) or Leucine (L) or Isoleucine (I) or Methionine (M) or Cysteine (C)
Sulphur-containing side chain —Cysteine (C) or Methionine (M)
Aromatic side chain —Phenylalanine (F) or Tyrosine (Y) or Tryptophane (W)
Neutral side chain—Glutamine (Q) or Asparagine (N) or Histidine (H)
Acidic side chain —Glutamate (E) or Aspartate (D), or Histidine (H)
Basic side chain —Lysine (K) or Arginine (R).

22. The process of claim 18 wherein said subsequence 3 begins with a di- or tri-peptide motif of amino acid residues comprising glutamate (E) or glutamine (Q) or phenylalanine (F) or lysine (K) or alanine (A) or leucine (L).

23. The process of claim 18 wherein said subsequence 3 begins with a proline (P) positioned within three amino acid residues of a di- or tri-peptide motif of amino acid residues comprising glutamate (E) or glutamine (Q) or phenylalanine (F) or lysine (K) or alanine (A) or leucine (L).

24. A peptide produced according to the process of claim 18 wherein said subsequence 3 contains a concentration of said Helical amino acids in excess of about 40%.

25. A peptide produced according to the process of claim 18 wherein said subsequence 3 is uniformly constructed from said Helical amino acids.

26. A peptide produced according to the process of claim 18 wherein said subsequence 3 terminates with alanine (A), glutamate (E), glutamine (Q), tyrosine (Y), phenylalanine (F), lysine (K) or proline (P) residues.

27. A peptide produced according to the process of claim 18 wherein said subsequence 3 is of minimum length of 6 amino acid residues which subsequence is conserved across related viral family members.

28. A peptide produced according to the process of claim 18 further comprising one or more adducts at either the amino- or carboxy-termini of said peptide.


SEQUENCE LISTING (52 sequences)

SEQ ID NO: 1 - protein, 67 aa, Human metapneumovirus
YQLSKVEGEQHVIKGRPVSSSFDPIKFPEDQFNVALDQVFESIENSQALVDQSNKILNSAEKGNTGF

SEQ ID NO: 2 - protein, 78 aa, Human coronavirus
PELDSFKEELDKYFKNHTSPDVDLGDISGINASVVNIQKEIDRLNEVAKNLNESLIDLQELGKYEQYIKWPWYVWLGF

SEQ ID NO: 3 - protein, 34 aa, Human metapneumovirus
YQLSKVEGEQHVIKGRPVSSSFDPIKFPEDQFNV

SEQ ID NO: 4 - protein, 33 aa, Human metapneumovirus
PVSSSFDPIKFPEDQFNVALDQVFESIENSQAL

SEQ ID NO: 5 - protein, 33 aa, Human metapneumovirus
ALDQVFESIENSQALVDQSNKILNSAEKGNTGF

SEQ ID NO: 6 - protein, 28 aa, Human metapneumovirus
YQLSKVEGEQHVIKGRPVSSSFDPIKFP

SEQ ID NO: 7 - protein, 39 aa, Human metapneumovirus
EDQFNVALDQVFESIENSQALVDQSNKILNSAEKGNTGF

SEQ ID NO: 8 - protein, 37 aa, Human metapneumovirus
KFPEDQFNVALDQVFESIENSQALVDQSNKILNSAEK

SEQ ID NO: 9 - protein, 34 aa, Human metapneumovirus
EDQFNVALDQVFESIENSQALVDQSNKILNSAEK

SEQ ID NO: 10 - protein, 36 aa, Human coronavirus
PELDSFKEELDKYFKNHTSPDVDLGDISGINASVVN

SEQ ID NO: 11 - protein, 37 aa, Human coronavirus
DVDLGDISGINASVVNIQKEIDRLNEVAKNLNESLID

SEQ ID NO: 12 - protein, 36 aa, Human coronavirus
RLNEVAKNLNESLIDLQELGKYEQYIKWPWYVWLGF

SEQ ID NO: 13 - protein, 26 aa, Human coronavirus
NIQKEIDRLNEVAKNLNESLIDLQEL

SEQ ID NO: 14 - protein, 28 aa, Human coronavirus
LNESLIDLQELGKYEQYIKWPWYVWLGF

SEQ ID NO: 15 - protein, 20 aa, Human coronavirus
QELGKYEQYIKWPWYVWLGF

SEQ ID NO: 16 - protein, 15 aa, Human coronavirus
YEQYIKWPWYVWLGF

SEQ ID NO: 17 - protein, 14 aa, Human coronavirus
YEQYIKWPWYVWLG

SEQ ID NO: 18 - protein, 10 aa, Human coronavirus
YIKWPWYVWL

SEQ ID NO: 19 - protein, 20 aa, Human coronavirus
PELDSFKEELDKYFKNHTSP

SEQ ID NO: 20 - protein, 73 aa, Human coronavirus
PNLPDFKEELDQWFKNQTSVAPDLSLDYINVTLDLQVEMNRLQEAIKVLNQSYINLKDIGTYEYYVKWPWYVW

SEQ ID NO: 21 - protein, 27 aa, Human coronavirus
PNLPDFKEELDQWFKNQTSVAPDLSLD

SEQ ID NO: 22 - protein, 42 aa, Human coronavirus
YINVTFLDLQVEMNRLQEAIKVLNQSYINLKDIGTYEYYVKW

SEQ ID NO: 23 - protein, 33 aa, Human coronavirus
QVEMNRLQEAIKVLNQSYINLKDIGTYEYYVKW

SEQ ID NO: 24 - protein, 28 aa, Human coronavirus
QEAIKVLNQSYINLKDIGTYEYYVKWPW

SEQ ID NO: 25 - protein, 20 aa, Human coronavirus
QSYINLKDIGTYEYYVKWPW

SEQ ID NO: 26 - protein, 12 aa, Human coronavirus
YEYYVKWPWYVW

SEQ ID NO: 27 - protein, 31 aa, Mouse hepatitis virus
QDAIKKLNESYINLKEVGTYEMYVKWPWYVW

SEQ ID NO: 28 - protein, 6 aa, Human immunodeficiency virus type 1
FLGFLG

SEQ ID NO: 29 - protein, 4 aa, Human coronavirus
TTTS

SEQ ID NO: 30 - protein, 5 aa, Human coronavirus
ELDKY

SEQ ID NO: 31 - protein, 5 aa, Human immunodeficiency virus type 1
ELDKW

SEQ ID NO: 32 - protein, 3 aa, Human coronavirus
PEL

SEQ ID NO: 33 - protein, 5 aa, Mouse hepatitis virus
PDFKE

SEQ ID NO: 34 - protein, 7 aa, Human coronavirus
FKEELDK

SEQ ID NO: 35 - protein, 8 aa, Human coronavirus
KWPWYVWL

SEQ ID NO: 36 - protein, 6 aa, Human metapneumovirus
QALVDQ

SEQ ID NO: 37 - protein, 5 aa, Artificial
  This is an artificially created peptide acetyl
PEQLK

SEQ ID NO: 38 - protein, 11 aa, Human immunodeficiency virus type 1
GRKKRRQRRRP

SEQ ID NO: 39 - protein, 23 aa, Homo sapiens
ELRVRLASHLRKLRKRLLRDADD

SEQ ID NO: 40 - protein, 7 aa, Mouse hepatitis virus
RIQDAIK

SEQ ID NO: 41 - protein, 7 aa, Human coronavirus
RLNEVAK

SEQ ID NO: 42 - protein, 21 aa, Human coronavirus
ENQKQIANQFNKAISQIQESL

SEQ ID NO: 43 - protein, 20 aa, Human coronavirus
KVQDVVNQNAQALNTLVKQL

SEQ ID NO: 44 - protein, 13 aa, Human coronavirus
KYEQYIKWPWYVW

SEQ ID NO: 45 - protein, 13 aa, Mouse hepatitis virus
TYEMYVKWPWYVW

SEQ ID NO: 46 - protein, 13 aa, Human coronavirus
TYEYYVKWPWYVW

SEQ ID NO: 47 - protein, 13 aa, Artificial
  SEQ ID NO 44 was arbitrarily scrambled to generate this sequence
YEWKWIYWYPVKQ

SEQ ID NO: 48 - protein, 83 aa, Human coronavirus
PNLPDFKEELDQWFKNQTSVAPDLSLDYINVTFLDLQVEMNRLQEAIKVLNQSYINLKDIGTYEYYVKWPWYVWLLICLAGVA

SEQ ID NO: 49 - protein, 85 aa, Mouse hepatitis virus
PNPPDFKEELDKWFKNQTSIAPDLSLDFEKLNVTLLDLTYEMNRIQDAIKKLNESYINLKEVGTYEMYVKWPWYVWLLIGLAGVA

SEQ ID NO: 50 - protein, 84 aa, Human coronavirus
PELDSFKEELDKYFKNHTSPDVDLGDISGINASVVNIQKEIDRLNEVAKNLNESLIDLQELGKYEQYIKWPWYVWLGFIAGLIA

SEQ ID NO: 51 - protein, 19 aa, Human coronavirus
WTFGAGAALQIPFAMQMAY

SEQ ID NO: 52 - protein, 36 aa, Mouse hepatitis virus
RIQDAIKKLNESYINLKEVGTYEMYVKWPWYVWLLI